(19) United States
(12) Patent Application Publication
(10) Pub. No.: US 2020/0353057 A1
(43) Pub. Date: Nov. 12, 2020

(54) REDUCING IMMUNOGENICITY TO PEGLOTICASE

(71) Applicant: Horizon Pharma Rheumatology LLC, Lake Forest, IL (US)

(72) Inventors: Jeffrey KENT, Deerfield, IL (US); Brian LAMOREAUX, Lake Forest, IL (US)

(21) Appl. No.: 16/942,569

(22) Filed: Jul. 29, 2020

Related U.S. Application Data
(63) Continuation-in-part of application No. PCT/US2020/015958, filed on Jan. 30, 2020.
(60) Provisional application No. 62/903,567, filed on Sep. 20, 2019.
(60) Provisional application No. 62/798,783, filed on Jan. 30, 2019.

(51) Int. Cl. A61K 38/44; A61K 31/365; A61K 9/00; A61K 47/60; A61P 19/06

(52) U.S. Cl. CPC A61K 38/44; A61K 31/365; A61P 19/06; A61K 47/60; A61K 9/0053

(57) ABSTRACT

The disclosure provides methods of treating gout in patients comprising administering a PEGylated uricase. Also provided are methods of treating gout in patients comprising co-administering a PEGylated uricase and MMF. Also provided are methods of reducing intolerance to a PEGylated uricase and prolonging the urate lowering effect comprising co-administration of the PEGylated uricase and MMF.

### CROSS REFERENCE TO RELATED APPLICATIONS

This application is a continuation-in-part of International Application No. PCT/US2020/015958, filed Jan. 30, 2020, which claims the benefit of U.S. Provisional Patent Application No. 62/798,783, filed Jan. 30, 2019 and U.S. Provisional Patent Application No. 62/903,567 filed Sep. 20, 2019, each of which is incorporated by reference herein in its entirety.

### FIELD OF THE DISCLOSURE

Gout affects approximately 4% of the U.S. population, is the most common form of chronic inflammatory arthritis in men older than 40 years of age, and is associated with decreased quality of life. The frequency of gout is increasing worldwide, with prevalence rates estimated to be as high as 7% in older men, largely associated with aging, obesity, and people living longer with chronic illness. Although it is estimated that up to 400,000 (up to 5% of the estimated 8 million persons with gout) in the United States experience chronic refractory gout, characterized by ongoing symptoms of active disease and a failure to control/maintain serum urate (SUA) <6 mg/dL with conventional xanthine oxidase inhibitors (i.e. allopurinol and febuxostat) and uricosuric agents (i.e. probenecid), gout is vastly undertreated by primary care providers, and 33% of gout patients are on urate-lowering therapy (Chen-Xu 2019). These patients often have significant, disabling urate deposits in soft tissues and bone known as tophi. Gout patients have a significantly higher incidence of emergency room visits, hospitalizations, outpatient visits, and other medical services, a significant medical burden compared to patients without gout.

Uric acid (UA) is the end metabolite in the human purine catabolic pathway. When the concentration of serum uric acid (SUA) is above the biochemical limit of solubility, 6.8 mg/dL, monosodium urate crystals may precipitate in tissues. It is hypothesized that after many years of persistent hyperuricemia, accumulation of monosodium urate crystals causes symptoms of gout, such as acute inflammation of joints (gout flare), formation of gout tophi, gouty arthritis, and UA nephropathy (including UA renal stones). Control of chronic gout cannot be achieved without maintaining SUA <6 mg/dL. A total of 9.2 million patients have been diagnosed with gout in the United States (Chen-Xu, 2019). The principal pharmaceutical approach to the treatment of gout is the use of the xanthine oxidase inhibitors, allopurinol, and febuxostat, to block the synthesis of UA. Approximately 2% of patients treated with allopurinol develop allergic reactions and a severe hypersensitivity syndrome occurs in about 0.4% of the patients. Patients with medical contraindications to xanthine oxidase inhibitors because of allergy/hypersensitivity, or who have failed to normalize SUA at maximum medically appropriate doses of these medications, can go on to develop chronic gout.

Pegloticase or PEGylated uricase (KRYSTEXXA®; “KXX”) is a monomethoxypoly(ethylene glycol) (PEG) modified recombinant mammalian uricase (urate oxidase) which reduces levels of UA in the serum (or plasma) by enzymatically degrading urate, i.e., catalyzing its conversion to allantoin, a water-soluble metabolite more readily excreted in the urine than uric acid. Pegloticase provides a new therapeutic mechanism to reduce SUA in patients with chronic gout refractory to conventional oral therapy. These patients experience a severe burden of gout disease characterized by tophi (approximately 70%), frequent and often crippling flares (approximately 7 per year), and deforming arthritis. Pegloticase provides medical benefits in patients who respond by lowering SUA and by reducing tophus burden in these patients who currently have no therapeutic options.

Seven clinical studies have been conducted with pegloticase in patients with refractory chronic gout. The Phase 1 program established an acceptable profile of tolerability and safety for intravenous (IV) dosing, whereas subcutaneous dose administration was less well tolerated. The Phase 2 program identified a minimally effective dose (4 mg), a dose-response plateau dose (12 mg), a safe and optimally effective dose (8 mg), and a once every 2 weeks or once every 4 weeks dosing regimen.

Two randomized, double-blind, placebo-controlled, multi-center, 6-month safety and efficacy Phase 3 studies have been conducted in a total of 225 hyperuricemic patients (SUA >8 mg/dL) with symptomatic gout who reported contraindication to or who had failed to normalize SUA with allopurinol therapy. The pooled efficacy results showed improvements in tophus burden consistent with urate-lowering effect of pegloticase in both dose groups. Improvements were more rapid in patients who received pegloticase 8 mg every 2 weeks compared to every 4 weeks and met the outcomes data of complete resolution of at least 1 tophus with no new or progressive tophi as assessed by blinded assessment of digital photographs of target tophi.

The pooled safety results from these Phase 3 studies showed that gout flares were more common in the pegloticase groups than in the placebo group during the first 3 months of therapy, a physiological effect resulting from SUA-lowering which is commonly observed upon the initiation of all urate-lowering therapies. During the second 3 months of treatment, a lower proportion of pegloticase-treated patients experienced flares than patients receiving placebo. The incidence of flares during this time period was lowest in the group receiving pegloticase 8 mg every 2 weeks than in the group who received pegloticase 8 mg every 4 weeks, as was the incidence of infusion-related reactions (26% with biweekly dosing vs. 40% with the every-4-week dosing regimen). Both infusion reactions (IRs) and gout flares were least common in patients with sustained urate-lowering responses to treatment and those who received bi-weekly treatment. In most pegloticase-treated patients with IRs, a loss of response to pegloticase (return to SUA ≥6 mg/dL) preceded the time of the first IR (20/21; 95%).

A relationship between the loss of urate-lowering efficacy, incidence of IRs, and high-titer antibody formation was identified in a post-hoc analysis of the pooled data from the Phase 3 studies. Patients with high anti-pegloticase antibody titers (>1:2430) showed a loss of pegloticase activity attributed to a more rapid clearance of drug in the presence of these antibodies. In one study, 69 (41%) of 169 patients receiving pegloticase developed high titer anti-pegloticase antibodies and subsequently lost response to the drug. In a second study, only 1 of 52 participants with high antibody titers maintained a response to pegloticase (serum urate <6 mg/dL). In addition, 60% participants with high titers developed IR. Anti-pegloticase antibodies were largely directed to the polyethylene glycol (PEG) portion of the molecule and altered the pharmacokinetic clearance of pegloticase, resulting in inhibition of SUA lowering activity. In another study, only 7 of 65 patients (10.8%) with an antibody titer exceeding 1:2430 at any time during treatment maintained a response to pegloticase compared with 89.2% (58/65) who had never had an antibody titer above that level. In addition, 31 of 52 (60%) patients with titers exceeding 1:2430 developed IRs. The ability of pegloticase to induce antibody production demonstrated the antigenic potential of the drug, and thus raised the possibility that relatively large or more frequent doses of pegloticase (antigen) might reduce antibody formation by induction of antigen-specific non-responsiveness (high zone tolerance). By preventing the formation of anti-pegloticase antibodies, a dose regimen should prevent loss of response to the drug and decrease the incidence of IRs associated with it.

As described herein, an alternate approach to prevent immunogenicity by pegloticase and therefore reduce the incidence of IRs, is co-administration of pegloticase and an immunosuppressive agent. As described herein, one such immunosuppressive agent is mycophenolate mofetil (MMF) immunosuppressive therapy.

The long-term safety of pegloticase has been demonstrated in an open-label extension study that enrolled 151 patients: 149 received pegloticase either bi-weekly or every 4 weeks for up to 30 months and 2 chose observation only. No new safety signals were observed and ongoing patient benefit in a number of clinical outcome measures was maintained beyond the 6-month period of the double-blind studies.

In one aspect, the disclosure provides a method of treating gout in a patient having a serum uric acid level of ≥6 mg/dL comprising: administering MMF to said patient at a dose of 500 mg twice per day orally for a period of 2 weeks prior to the first administration of a PEGylated uricase; co-administering the PEGylated uricase and MMF to said patient using a dosage regimen comprising a dose of 8 mg of the PEGylated uricase intravenously every 2 weeks for a total of 12 doses; and a dose of 1000 mg MMF twice per day orally, wherein the co-administered MMF is administered concurrently with each administration of the PEGylated uricase; administering 8 mg of the PEGylated uricase at a dosage of 8 mg intravenously every 2 weeks for a total of 12 doses.

In another aspect, the disclosure provides a method of reducing or preventing loss of a response to a PEGylated uricase and prolonging the urate lowering effect comprising co-administration of the PEGylated uricase at a dosage of 8 mg intravenously every 2 weeks and MMF at a dosage of 1000 mg twice per day orally to a patient having a serum uric acid level of ≥6 mg/dL prior to PEGylated uricase treatment initiation; wherein the administration of the PEGylated uricase and MMF result in the serum uric acid level being normalized relative to a patient not receiving co-administration of the PEGylated uricase and MMF immunosuppressive therapy.

In one embodiment, a method as described herein further comprises a prophylactic regimen of colchicine for a period of at least 2 weeks prior to the first administration of the PEGylated uricase. In another embodiment, wherein the SUA levels of the patient are determined prior to each dose of the PEGylated uricase. In another embodiment, such a method further comprises measuring one or more of trough PEGylated uricase levels, anti-PEGylated uricase antibody levels, and anti-PEG antibody levels, prior to each dose of the PEGylated uricase after the first dose.

In another embodiment, any of such methods described herein further comprises measuring serum uric acid (SUA) levels, hematology, and/or liver function tests on a weekly basis or every other week during treatment. In another embodiment, blood tests may be performed every other week, or every third week. For example, blood tests may be performed at weeks 3, 5, 7, 9, 11, 13, 15, 17, 19, 21, 23. In some embodiments, a blood test as described herein may be performed on the same day each week, or may be performed within 1 or 2 days of the previous blood test performed. Blood tests may be drawn for any blood tests described herein within 24 hours prior to administration of KXX.

For determination of serum uric acid, if the SUA is ≥6 mg/dL, the subject may not be dosed and the SUA may be repeated. In some embodiments, if the SUA is ≥6 mg/dL, a subject may be withdrawn from the study. In other embodiments, samples that result in discordant results between laboratories may be evaluated to discuss whether the subject should continue on study drug or discontinue dosing.

In another embodiment, said co-administration of the PEGylated uricase and MMF results in normalization of the serum uric acid level in the patient relative to a patient not receiving co-administration of the PEGylated uricase and MMF. In another embodiment, the serum uric acid level is reduced to less than 6 mg/dL as a result of co-administration of the PEGylated uricase and MMF. In another embodiment, the serum uric acid level is reduced to less than 5 mg/dL as a result of co-administration of the PEGylated uricase and MMF. In another embodiment, the serum uric acid level is reduced to less than 2 mg/dL as a result of co-administration of the PEGylated uricase and MMF.

In another embodiment, the incidence of infusion reaction, gout flare, or anaphylaxis is reduced as a result of co-administration of the PEGylated uricase and MMF. In another embodiment, the level of MMF metabolite is increased relative to a patient not receiving co-administration of the PEGylated uricase and MMF.

In another embodiment, such a method further comprises measuring one or more of peripheral joint urate deposition volume and inflammatory volume. In another embodiment, peripheral joint urate deposition volume is reduced in the patient relative to a patient not receiving co-administration of the PEGylated uricase and MMF treatment. In another embodiment, peripheral joint urate deposition volume is determined by dual-energy computed tomography (DECT) scanning.

In another embodiment, inflammatory volume is reduced in the patient relative to a patient not receiving co-administration of the PEGylated uricase and MMF treatment. In another embodiment, inflammatory volume is determined by Dynamic Contrast Enhanced-Magnetic Resonance Imaging (DCE-MRI) or MRI without contrast, or both.

In another embodiment, the mean titer of anti-PEGylated uricase antibodies is less than or equal to 1:7000 as a result of co-administration of the PEGylated uricase and MMF treatment. In another embodiment, the serum uric acid level is normalized by week 12 after co-administration of the PEGylated uricase and MMF treatment begins.

These and other embodiments of the disclosure are described in detail below.

### BRIEF DESCRIPTION OF THE DRAWINGS

FIG. 1 shows a graph of the urate reducing efficacy of pegloticase.

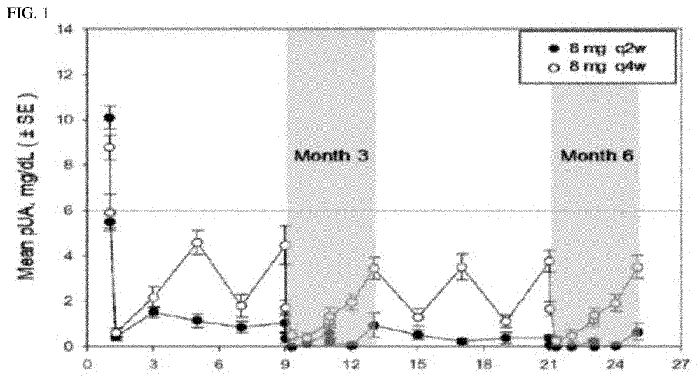

FIG. 2 shows the patient level timeline for the present study.

FIG. 3 shows a decision table for pursuing future clinical trials.

FIG. 4 shows the primary outcome for study participants.

### DETAILED DESCRIPTION

Overview

In one embodiment, the disclosure provides a method of treating gout in a patient having a serum uric acid level of ≥6 mg/dL comprising: administering MMF to said patient at a dose of 500 mg twice per day orally for a period of 2 weeks prior to the first administration of a PEGylated uricase; co-administering the PEGylated uricase and MMF to said patient using a dosage regimen comprising a dose of 8 mg of the PEGylated uricase intravenously every 2 weeks for a total of 12 doses; and a dose of 1000 mg MMF twice per day orally, wherein the co-administered MMF is administered concurrently with each administration of the PEGylated uricase; administering 8 mg of the PEGylated uricase at a dosage of 8 mg intravenously every 2 weeks for a total of 12 doses. In another embodiment, the disclosure provides a method of reducing or preventing loss of response to the PEGylated uricase and prolonging the urate lowering effect comprising co-administration of the PEGylated uricase at a dosage of 8 mg intravenously every 2 weeks and MMF at a dosage of 1000 mg twice per day orally to a patient having a serum uric acid level of ≥6 mg/dL prior to PEGylated uricase treatment initiation; wherein the administration of the PEGylated uricase and MMF result in the serum uric acid level being normalized relative to a patient not receiving co-administration of the PEGylated uricase and MMF immunosuppressive therapy.

### KRYSTEXXA® (Pegloticase)

Pegloticase or PEGylated uricase (KRYSTEXXA®; “KXX,”) is a uric acid specific enzyme, which is a PEGylated product that consists of recombinant modified mammalian urate oxidase (uricase) produced by a genetically modified strain of Escherichia coli. KXX is indicated for the treatment of chronic gout in patients refractory to conventional therapy. KXX® is described at least in U.S. Pat. Nos. 8,188,224; 7,811,800; 9,534,013; 6,576,235; 9,377,454; 6,783,965; as well as PCT Publ. No. WO 2018/089808, each of which is incorporated herein in its entirety. In some embodiments, non-mammalian uricases may be used as deemed appropriate, or a uricase from any species. In other embodiments, muteins of a uricase as described herein, having an altered amino acid sequence, may be used and are encompassed within the present disclosure.

Certain uricases are useful for preparing conjugates with various forms of poly(ethylene glycol) or poly(ethylene oxide) (both referred to as PEG) to produce therapeutically efficacious forms of uricase having increased protein half-life and reduced immunogenicity. Thus, in some embodiments, uricase is covalently conjugated to monomethoxypoly(ethylene glycol) [mPEG] (10 kDa molecular weight). The cDNA coding for uricase is based on mammalian sequences. Each uricase subunit has a molecular weight of approximately 34 kDa per subunit. The average molecular weight of pegloticase (tetrameric enzyme conjugated to mPEG) is approximately 540 kDa.

In some embodiments, a uricase as described herein may be conjugated to any desired number of PEG or mPEG molecules, such as 1, 2, 3, 4, 5, 6, 7, 8, 9, 10, 11, 12, 13, 14, or the like. In other embodiments, a uricase as described herein may be conjugated to other modifiers in addition to, or alternatively to, PEG or mPEG. Such PEG or mPEG molecules may be attached to a uricase using any means appropriate in accordance with the disclosure. For example, a PEG or mPEG molecule may be conjugated to a uricase as described herein by a cysteine residue, or a serine residue, or a lysine residue. A PEG or mPEG may be attached to a uricase as described herein using any specific amino acid in accordance with the disclosure.

In other embodiments, a uricase of the present disclosure may be modified with a non-PEG modification. For example, one or more residues of proline, alanine, and/or serine (PAS), or combinations thereof, referred to herein as PASylation. In other embodiments, a uricase as described herein may be modified by conjugation with an antibody, a protein, or a small molecule, or may be conjugated to poly(2-ethyl-2-oxazoline) referred to herein as POZylation. In other embodiments, a uricase may be modified at the amine end or the carboxy end, or both. In other embodiments, any other modifiers deemed appropriate may be used to extend the half-life in curculation in accordance with the present disclosure.

Mode of Action of KXX

KXX achieves its therapeutic effect by catalyzing the oxidation of uric acid to allantoin, thereby lowering serum uric acid. Allantoin is an inert and water-soluble purine metabolite. It is readily eliminated, primarily by renal excretion.

KXX (pegloticase) concentrations are expressed as concentrations of uricase protein. Each mL of KXX contains 8 mg of uricase protein (conjugated to 24 mg of 10 kDa mPEG), 2.18 mg Disodium Hydrogen Phosphate Dihydrate (Na₂HPO₄.2H₂O), 8.77 mg Sodium Chloride (NaCl), 0.43 mg Sodium Dihydrogen Phosphate Dihydrate (NaH₂PO₄.2H₂O), and Water for Injection to deliver 8 mg of pegloticase (as uricase protein).

KXX was granted orphan designation by the FDA on Feb. 21, 2001 (ODA #00-1356) and KXX 8 mg every 2 weeks by IV infusion was approved by the United States (US) FDA on Sep. 14, 2010 for the treatment of adult patients with chronic gout refractory to conventional therapy. Since pegloticase was approved in the US, there have been no new safety signals reported to Horizon Pharma, PLC (Horizon) the manufacturer of KXX. The most common adverse events continue to be IRs, anaphylaxis, and gout flares. Post-marketing safety information suggests that the concomitant use of pegloticase with urate-lowering agents may mask the detection of patients who have lost therapeutic response to the drug and increase the risk of IR and/or anaphylaxis. Pegloticase is contraindicated in patients with glucose-6-phosphate dehydrogenase (G6PD) deficiency because of the risk of hemolysis and methemoglobinemia.

Treatment of Patients with KXX

KXX treatment may be initiated with monitoring of serum uric acid (SUA) levels prior to each infusion. In some embodiments, KXX therapy may be discontinued if the SUA levels increase to 6 mg/dL or above, particularly when 2 consecutive levels of 6 mg/dL or above are observed.

In addition, in order to reduce the incidence of infusion reactions, adverse events (AEs), such as gout flare, or serious adverse events (SAEs) such as anaphylaxis, patients may be pre-medicated with antihistamines and/or corticosteroids. AEs and SAEs are described in detail below. Anaphylaxis or other IRs may occur with any infusion, including a first infusion, or any subsequent infusion, and generally manifests within 2 hours of the infusion. Delayed-type hypersensitivity reactions may also occur. The most common adverse reactions (occurring in about 5% or more of KXX-treated patients) are gout flares, infusion reactions, nausea, contusion or ecchymosis, nasopharyngitis, constipation, chest pain, anaphylaxis, and vomiting. Additional monitoring of patients during and after infusion may be beneficial to prevent or detect such reactions. In some embodiments, patients are monitored for one hour or more following administration of KXX. In some embodiments, gout flare prophylaxis may be recommended for patients when treating with KXX. For example, gout flare prophylaxis may be recommended for a period of about the first six months of KXX therapy.

In some embodiments, patients or subjects receiving KXX therapy, either alone or co-administered with an immunosuppressive agent, may experience exacerbation of congestive heart failure. For such patients, close monitoring after infusion may be beneficial.

In some embodiments, and in order to prevent or manage reactions to KXX therapy, such as anaphylaxis and/or infusion reactions, KXX may be administered in a healthcare setting and by a healthcare provider. The KXX admixture may be administered by intravenous infusion over a minimum of 120 minutes via gravity feed, syringe-type pump, or infusion pump. As described in detail below, KXX may be administered alone to a patient, or may be co-administered to a patient or subject with an immunosuppressive agent such as mycophenolate mofetil (MMF). In some embodiments, KXX may be administered in a healthcare setting as described herein, and an immunosuppressive agent may be administered at home. In other embodiments, both KXX and an immunosuppressive agent such as MMF may be administered in a healthcare setting.

In some embodiments, pre-screening of patients or subjects to be administered KXX, either alone of co-administered with an immunosuppressive agent, such as MMF, for the presence of, or a risk for developing glucose-6-phosphate dehydrogenase (G6PD) deficiency may be beneficial. Such patients may be excluded from treatment with KXX because of a risk of hemolysis and/or methemoglobinemia.

In some embodiments, KXX, either alone, or in combination with an immunosuppressive agent or therapy, may be used to treat a patient with gout as described herein. In other embodiments, KXX may be used to treat other diseases involving the kidney, such as including, but not limited to, nephritis.

Dosage of KXX

The recommended dose and regimen of KXX for adult patients is 8 mg (uricase protein) given as an intravenous (IV) infusion every two weeks. The optimal treatment duration with KXX has not been established. KXX is a sterile, clear, colorless solution containing 8 mg/mL pegloticase in phosphate-buffered saline, and it intended for intravenous infusion.

Dosage Forms of KXX

KXX may be provided in a 1-mL sterile concentrate for dilution containing 8 mg of pegloticase protein, expressed in uricase protein amounts.

### Mycophenolate Mofetil (MMF)

It some embodiments, as described herein, KXX may be administered alone as a treatment, or may be co-administered with an immunosuppressive or immunomodulatory agent, such as MMF. In some embodiments, a short-term course of MMF can mitigate immunogenicity to pegloticase. In some embodiments, MMF suppresses formation of anti-pegloticase antibodies, reducing or eliminating the immune response. Development of anti-KXX antibodies may increase the clearance of KXX, thereby causing loss of a drug response in the patient. MMF is able to target the mechanism of pegloticase immunogenicity through inhibition of T and B cell proliferation. MMF is the pro-drug of active moiety mycophenolic acid, and is a potent, selective, and reversible inhibitor of inosine monophosphate dehydrogenase, the key enzyme of de novo purine synthesis in activated lymphocytes. The main adverse effects associated with oral MMF are gastrointestinal and hematologic (leukopenia) and are relatively mild in most participants.

To the Inventors' knowledge, this will be the first study to test the hypothesis that immunogenicity to pegloticase can be attenuated via MMF. For this study, a short course of the immune modulating agent MMF vs placebo (PBO) will be given to patients or subjects to improve treatment efficacy and reduce IR in patients being treated for chronic refractory gout as an innovative approach to gout management with pegloticase. New strategies to deal with the growing burden of gout and to improve use of existing therapies are urgently needed and the present disclosure represents a novel approach addressing both clinical and immunological questions.

In some embodiments, the dosage of MMF used in the present study is 500 mg twice per day, administered orally. In some embodiments, MMF may be administered at a dosage of 1000 mg twice per day, administered orally. Such a dosage may be administered to a patient to prevent or control immunogenicity to KXX or associated AEs, SAEs, or IRs occurring as a result of KXX treatment. In some embodiments, MMF may be administered to a patient starting when the serum uric acid level is 6 mg/dL or greater. Alternate dosages of MMF may be used as deemed appropriate by a clinician. For example, MMF may be administered at a dosage of 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg. 750 mg, 775 mg, 800 mg, 825 mg, 850 mg, 875 mg, 900 mg, 925 mg, 950 mg, 975 mg, 1000 mg, 1025 mg, 1050 mg, 1075 mg, 1100 mg, 1125 mg, 1150 mg, 1175 mg, 1200 mg, 1225 mg, 1250 mg, 1275 mg, 1300 mg, or the like. One of skill in the art will understand that dosages of drugs as described herein may be altered as needed for a patient or subject without deviating from the scope of the disclosure.

In other embodiments, MMF may be administered once per day, twice per day, 3 times per day, or more times per day as needed. In other embodiments, MMF may be administered every 2 days, or every 3 days, or every 4 days, or every 5 days, or every 6 days, or every 7 days, or every 8 days, or every 9 days, or every 10 days, or every 11 days, or every 12 days, or every 13 days, or every 14 days. In other embodiments, the drug may be administered one per week, twice per week, 3 times per week, 4 times per week, 5 times per week, 6 times per week, 7 times per week, 8 times per week, 9 times per week, 10 times per week, 11 times per week, 12 times per week, 13 times per week, 14 times per week, or the like. In some embodiments, different dosages or frequencies may be used on different days, as described herein.

Clinical Pharmacology of MMF

Mycophenolate mofetil is well absorbed following oral administration. Maximum serum radioactivity occurs at 1 to 2 hours after oral ³⁵S-mycophenolate mofetil and decays with a half-life of 5 hours. This is not an estimate of the half-life of mycophenolate mofetil itself, but is the decay rate for all ³⁵S-containing metabolites of the drug. Because of extensive metabolism, only a fraction of the radioactivity is present as mycophenolate mofetil. Usual doses produce blood levels of mycophenolate mofetil, and of mercaptopurine derived from it, which are low (<1 mcg/mL). Blood levels are of little predictive value for therapy since the magnitude and duration of clinical effects correlate with thiopurine nucleotide levels in tissues rather than with plasma drug levels. Mycophenolate mofetil and mercaptopurine are moderately bound to serum proteins (30%) and are partially dialyzable.

Mycophenolate mofetil is metabolized to 6-mercaptopurine (6-MP). Both compounds are rapidly eliminated from blood and are oxidized or methylated in erythrocytes and liver; no mycophenolate mofetil or mercaptopurine is detectable in urine after 8 hours. Activation of 6-mercaptopurine occurs via hypoxanthine-guanine phosphoribosyl transferase (HGPRT) and a series of multi-enzymatic processes involving kinases to form 6-thioguanine nucleotides (6-TGNs) as major metabolites. The cytotoxicity of mycophenolate mofetil is due, in part, to the incorporation of 6-TGN into DNA.

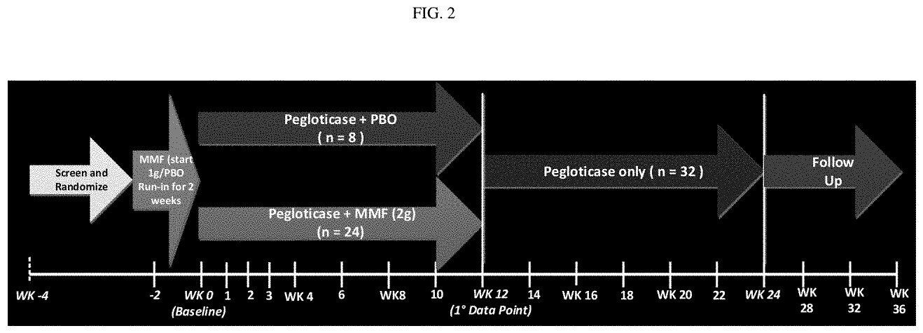

6-MP undergoes two major inactivation routes (see FIG. 2). One is thiol methylation, which is catalyzed by the enzyme thiopurine S-methyltransferase (TPMT), to form the inactive metabolite methyl-6-MP (6-MeMP). TPMT activity is controlled by a genetic polymorphism. For Caucasians and African Americans, approximately 10% of the population inherit one non-functional TPMT allele (heterozygous) conferring intermediate TPMT activity, and 0.3% inherit two TPMT non-functional alleles (homozygous) for low or absent TPMT activity. Non-functional alleles are less common in Asians. TPMT activity correlates inversely with 6-TGN levels in erythrocytes and presumably other hematopoietic tissues, since these cells have negligible xanthine oxidase (involved in the other inactivation pathway) activities, leaving TPMT methylation as the only inactivation pathway. Patients with intermediate TPMT activity may be at increased risk of myelotoxicity if receiving conventional doses of IMURAN®.

Patients with low or absent TPMT activity are at an increased risk of developing severe, life-threatening myelotoxicity if receiving conventional doses of IMURAN®. TPMT genotyping or phenotyping (red blood cell TPMT activity) can help identify patients who are at an increased risk for developing IMURAN® toxicity. Accurate phenotyping (red blood cell TPMT activity) results are not possible in patients who have received recent blood transfusions.

Another inactivation pathway is oxidation, which is catalyzed by xanthine oxidase (XO) to form 6-thiouric acid. The inhibition of xanthine oxidase in patients receiving allopurinol (ZYLOPRIM®) is the basis for the mycophenolate mofetil dosage reduction required in these patients.

Proportions of metabolites are different in individual patients, and this presumably accounts for variable magnitude and duration of drug effects. Renal clearance is probably not important in predicting biological effectiveness or toxicities, although dose reduction is practiced in patients with poor renal function.

### Pharmacokinetic (PK) Analysis

In some embodiments, patients or subjects of the present disclosure may have blood samples taken for PK analysis during treatment with KXX, either alone, or co-administered with an immunosuppressive or immunomodulatory agent, such as MMF. Although limited PK results have been reported in subjects receiving pegloticase, and no PK studies have been performed in subjects weighing ≥120 kg, the present disclosure provides PK analysis in patients receiving KXX treatment, either alone or co-administered with an immunosuppressive agent or therapy. Sundy et al. (JAMA 306(7):711-20, 2011) reported results from 24 subjects with refractory gout who received single doses from 0.5 to 12 mg of pegloticase. PK parameters included plasma uricase activity (pUox) and the plasma urate concentration (pUAc). In this study, the pUox half-life was 6.4 to 13.8 days. After doses of 4 to 12 mg, the pUAc fell within 24 to 72 hours, from a mean±SD value of 11.1±0.6 mg/dL to 1.0±0.5 mg/dL; the AUC value for the pUAc was equivalent to maintaining the pUAc at 1.2 to 4.7 mg/dL for 21 days post-infusion. It remains uncertain whether body mass affects drug distribution. Since pegloticase is administered as a single dose regardless of body mass, this may be beneficial to assess.

### Joint Imaging

In some embodiments, joint imaging may be performed for patients or subjects receiving KXX, either alone or co-administered with immunosuppressive therapy, such as MMF. Clinical research has shown that measuring tophus volume alone in gout is incomplete as successful therapy also needs to be associated with a reduction in inflammation, chronic synovitis, acute flares and slowing the progression or even healing of bone erosion, and thus, application of all-inclusive measurements that can measure all parameters of the physiologic impact of urate deposition will allow for comprehensive assessment of chronic gout and the impact of treatment. In some embodiments, a sub-study may also be performed to assess the ability of DECT and DCE-MRI to measure treatment response to pegloticase in subjects with chronic refractory gout.

### Co-Administration of KXX and Immunosuppressive/Immunomodulatory Therapy

Immunogenicity in response to pegloticase therapy (anti-pegloticase antibodies) may give rise to low serum drug levels, loss of therapeutic response, poor drug survival, and/or adverse events (e.g., IR). The development of anti-drug antibodies can be influenced by drug- and treatment-related factors, as well as participant characteristics. A potential prophylactic strategy to manage anti-drug antibody response with biologic response modifiers is the co-administration of immune modulating therapy. Reduction of immunogenicity with concomitant administration of other biologic agents (e.g. methotrexate use with adalimumab, infliximab) has been attributed to two mechanisms: 1) an immune modulating effect downregulating B cell activation, differentiation, and immunoglobulin production, and 2) alteration in Fc gamma R-mediated clearance mechanisms leading to prolongation of the half-life of monoclonal antibodies.

In a randomized controlled trial (RCT) for lupus nephritis, patients were treated concomitantly with mycophenolate mofetil (MMF) and glucocorticoids, and randomized to receive either rituximab or placebo. Over the 78-week study period, the serious adverse event rate, including infections, were similar in both groups and did not result in differential or unexpected safety signals. The rate of serious infections (19.9/100 patient-years and 16.6/100 patient-years in the placebo and rituximab arms, respectively) in this combination immunotherapies study is relevant, since gout flares may be treated with glucocorticoids, which may also increase infection risk.

In an open-label trial, thirty participants received pegloticase every three weeks for five infusions to investigate Ab response. Seven of these participants (3 of whom were on MMF receiving doses ranging from 500-2000 mg per day) were organ transplant recipients. Only one out of seven organ transplant recipients had a sustained high titer Ab response to pegloticase. Thus, organ transplant recipients on immune modulating therapies may be less prone to developing anti-pegloticase Ab, but further investigation of safer shorter term immune modulating strategies to minimize anti-pegloticase Ab are needed.

Thus, in some embodiments, the addition of immune modulating therapy (i.e., co-administration of KXX with an immunosuppressive or immunomodulatory agent) with an induction regimen or loading dose provides additive benefit in abrogating immunogenicity associated with biologics. As described herein, the immune modulating agent, mycophenolate mofetil (MMF), is beneficial for use as a short course therapy to improve treatment efficacy and reduce IR in patients being treated for chronic gout with KXX.

Various embodiments of the disclosure provide for treatment of gout or gout related symptoms by administering KXX, either alone or co-administered with an immunosuppressive or immunomodulatory agent. In some embodiments, KXX may be administered alone to a patient for treatment of gout. In other embodiments, KXX may be co-administered with an immunosuppressant or immunomodulatory agent, such as MMF, for a combined immunosuppressive therapy. As used herein, “immunosuppressive agent” may also be referred to as “immunosuppressant” or “immunosuppressive therapy.” In some embodiments, an “immunosuppressant” may also be referred to herein as an “immunomodulatory agent.”

Administration of an immunosuppressive drug may reduce or eliminate any immune reactions that may occur in the patient. An immune reaction that may be encountered in a drug treatment as described herein may be an allergic reaction or any associated symptoms, including, but not limited to, hives, itching, nasal congestion, rash, scratchy throat, watery or itchy eyes. Severe allergic reactions may have additional symptoms, including, but not limited to abdominal cramping or pain, pain or tightness in the chest, intestinal upset, dizziness, nausea, weakness, or the like. In some embodiments, a severe allergic reaction may include symptoms of anaphylaxis as described herein. Drug reactions may be referred to herein as adverse events (AEs), and may be mild AEs or may be serious AEs (SAEs). Such combination of KXX and another drug, such as MMF, may reduce adverse events in a patient or subject. Thus, in some embodiments, administration of an immunosuppressive or immunomodulatory therapy with KXX may be beneficial for patients having gout or symptoms thereof. In some embodiments, a patient or subject may be an individual having a serum uric acid level of ≥6 mg/dL prior to PEGylated uricase treatment initiation.

In some embodiments, patients or subjects may benefit from a particular dosage of KXX, or a particular dosage regiment, as described herein. For example, patients may be treated with an initial dose of KXX, such as a dose of 8 mg KXX on a weekly basis for 3 weeks for a total of 3 doses. In some embodiments, such a tolerizing dosage may be altered as deemed necessary by a clinician or practitioner.

Alternatively, for patients weighing greater than or equal to 120 kg, a tolerizing dose of KXX may include a dose of 8 mg, 12 mg, or 16 mg KXX intravenously at the first week of treatment for a total of one dose, followed by 8 mg intravenously on a weekly basis for 2 weeks after the first week of treatment, for a total of 2 doses. Such alternate dosing may be beneficial for patients having greater body weight, or a higher body mass index (BMI). In some embodiments, patients weighing greater than or equal to 120 kg may be grouped into treatment groups, with each group receiving a different tolerizing dosage of KXX. For example, one group may receive a tolerizing dose of 8 mg KXX, while another group may receive a tolerizing dose of 12 mg KXX, and still another group may receive a tolerizing dose of 16 mg KXX.

In some embodiments, KXX may be administered to a patient following a tolerizing dose at a dosage such as 8 mg by intravenous infusion every 2 weeks. This dosage may be continued for any period of time deemed appropriate by a clinician or practitioner. For example, 8 mg KXX may be given to a particular patient every 2 weeks following a tolerizing dosage regimen and lasting for a period of time totaling 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, or the like. In some embodiments, a patient may be given to a patient for more than 6 months, or more than 7 months, or more than 8 months, or more than 9 months, or more than 10 months, or more than 11 months, or more than 12 months, or more than 18 months, or more than 24 months. In other embodiments, KXX may be given to a patient for any length of time deemed appropriate by a clinician or practitioner, and as described herein, for the remainder of the patient's life.

In some embodiments, the patient may be treated with KXX plus an immunosuppressive agent or therapy for at least one month. In some embodiments, the patient is treated for 2 months, 3 months, 4 months, 5 months, 6 months, 7 months, 8 months, 9 months, 10 months, 11 months, 12 months, 15 months, 18 months, 21 months, 24 months, 30 months, 36 months, or more. In some embodiments, the patient may be treated for up to 30 weeks, or 25 weeks, or 20 weeks, or 17 weeks, or 12 weeks, or 6 weeks, or 4 weeks, or 2 weeks, or 24 months, or up to 36 months, or up to 48 months. In some embodiments, the patient may be treated for more than 24 months. In some embodiments, the patient is treated for the rest of the patient's life.

In some embodiments, a tolerizing dosage regimen as described herein may be combined with the use or co-administration of an immunosuppressive agent or therapy. Such a tolerizing dosage regimen may involve escalation of a dose of KXX and an immunosuppressive agent or therapy such that the patient or subject is able to better tolerate KXX or the dosage thereof. In other embodiments, such a tolerizing dosage regimen may involve increasing or escalating doses of KXX with a particular dose of an immunosuppressive agent or therapy. Such treatments may be employed for any duration as described herein.

In some embodiments, treatment with KXX may be continued as appropriate for any length of time, as long as the patient experiences an improvement in the symptoms or signs of gout. Such signs/symptoms of gout that may serve as a metric for improvement in disease severity may include, but are not limited to, serum uric acid level, peripheral joint urate deposition volume and inflammatory volume. For example, KXX treatment, either alone, or co-administered with an immunosuppressive agent, such as MMF, may be monitored with regular assessment of the patient or subject before, during, and following treatment. Any number of diagnostic or evaluative testing procedures may be performed at any time, and at any frequency as deemed necessary by a clinician or practitioner.

KXX treatment typically is monitored using regular determination of the patient's SUA levels. A reduction in the serum uric acid level relative to the SUA in the patient before KXX treatment may generally be indicative of successful treatment with KXX, alone, or co-administered with an immunosuppressive agent or therapy, such as MMF, as described herein. Collection and measurement of a patient's serum uric acid levels are known to those of skill in the art. In some embodiments, the patient's serum uric acid levels are assayed before each KXX therapy. In some embodiments, any suitable method for collecting appropriate samples and methods of measuring or quantifying uric acid levels may be used in accordance with the disclosure.

Additional measurements to assess the efficacy and safety of KXX treatment may be used in accordance with the disclosure. These may include trough KXX levels, anti-KXX antibody levels, and/or anti-PEG antibody levels. In some embodiments, these measurements may be taken prior to each dose of KXX after the first dose. In other words, an initial dose of KXX may be administered to a patient without measurement of KXX levels, anti-KXX antibody levels, and/or anti-PEG antibody levels. Then, prior to each subsequent dose of KXX, such measurements may be taken as described herein. For treatment regiments wherein KXX is co-administered with an immunosuppressive agent or therapy, the same measurements may be taken at the same time periods, without deviating from the scope of the disclosure. Such measurements may be obtained from each patient as necessary in order to evaluate response to the treatment, or a lack thereof. Specific criteria for responders and non-responders to treatment with KXX are described in the Examples.

In some embodiments, when KXX is co-administered with an immunosuppressive agent or therapy, such as MMF, additional measurements for assessing the efficacy of treatment may be obtained. For example, trough MMF metabolite levels may be obtained for each patient or subject prior to each dose of KXX, as described herein. In other embodiments, measurement of hematology and liver function may be obtained for each patient on a weekly basis during treatment. This type of measurement may provide information to clinicians relating to the breakdown of the immunosuppressive agent in the patient's body.

In some embodiments, co-administration of KXX and mycophenolate mofetil immunosuppressive therapy results in normalization of the serum uric acid level in the patient relative to a patient not receiving KXX and mycophenolate mofetil immunosuppressive therapy. As used herein, “normalization” refers to lowering of the serum uric acid level similar to that found in normal healthy patients. In other embodiments, normalization may refer to SUA levels being reduced to levels similar to that found in patients not requiring KXX treatment or therapy. In some embodiments, the serum uric acid level is normalized by week 17 after KXX and mycophenolate mofetil immunosuppressive therapy begins.

In some embodiments, as described herein, the serum uric acid level in patients treated with KXX and an immunosuppressive therapy such as MMF is reduced to less than 6 mg/dL as a result of treatment, including, but not limited to, 5.9 mg/dL, 5.8 mg/dL, 5.7 mg/dL, 5.6 mg/dL, 5.5 mg/dL, 5.4 mg/dL, 5.3 mg/dL, 5.2 mg/dL, 5.1 mg/dL, 5 m/dL, 4.9 mg/dL, 4.8 mg/dL, 4.7 mg/dL, 4.6 mg/dL, 4.5 mg/dL, 4.4 mg/dL, 4.3 mg/dL, 4.2 mg/dL, 4.1 mg/dL, 4 mg/dL, 3.9 mg/dL, 3.8 mg/dL, 3.7 mg/dL, 3.6 mg/dL, 3.5 mg/dL, 3.4 mg/dL, 3.3 mg/dL, 3.2 mg/dL, 3.1 mg/dL, 3 mg/dL, 2.9 mg/dL, 2.8 mg/dL, 2.7 mg/dL, 2.6 mg/dL, 2.5 mg/dL, 2.4 mg/dL, 2.3 mg/dL, 2.2 mg/dL, 2.1 mg/dL, 2 mg/dL, 1.9 mg/dL, 1.8. mg/dL, 1.7 mg/dL, 1.6 mg/dL, 1.5 mg/dL, 1.4 mg/dL, 1.3 mg/dL, 1.2 mg/dL, 1.1 mg/dL, 1 mg/dL, or the like. In other embodiments, the serum uric acid level may be reduced to less than 5 mg/dL as a result of treatment. In still further embodiments, the serum uric acid level is reduced to less than 2 mg/dL as a result of KXX and mycophenolate mofetil immunosuppressive therapy. Treatment with KXX plus an immunosuppressive therapy may be able to reduce the serum uric acid levels to levels that result in improvement of symptoms associated with gout as described herein.

In some embodiments, treatment of a patient or subject with KXX plus an immunosuppressive or immunomodulatory agent such as MMF results in a reduction of the incidence of infusion reaction, gout flare, or anaphylaxis.

In some embodiments, the level of mycophenolate mofetil metabolite is increased relative to a patient not receiving KXX and mycophenolate mofetil immunosuppressive therapy.

In some embodiments, analysis of the efficacy of a method as described herein for treating gout may further comprise measurements to assess disease severity. For example, a diagnostic imaging test, such as including, but not limited to, computed tomography (CT), magnetic resonance imaging (MRI), X-ray, ultrasound, positron emission tomography (PET), fluoroscopy, or the like.

In some embodiments, peripheral joint urate deposition volume and inflammatory volume may be measured in a patient and used to evaluate disease severity or to evaluate efficacy of the drug treatment. For example, in some embodiments, peripheral joint urate deposition volume is reduced in the patient relative to a patient not receiving KXX and mycophenolate mofetil immunosuppressive therapy. In other embodiments, peripheral joint urate deposition volume is determined by dual-energy computed tomography (DECT) scanning. In other embodiments, inflammatory volume is reduced in the patient relative to a patient not receiving KXX and mycophenolate mofetil immunosuppressive therapy. In other embodiments, inflammatory volume is determined by Dynamic Contrast Enhanced-Magnetic Resonance Imaging (DCE-MRI) or MRI without contrast, or both.

In some embodiments, administration of KXX alone or co-administration of KXX with an immunosuppressive therapy or agent or with a drug having monomethoxypoly(ethylene glycol) (PEG) may elicit an immune reaction in the patient or subject. Evaluation of antibodies in a subject or patient receiving KXX therapy may provide an assessment of such an immune reaction to the drug treatment. For example, in some embodiments, determination of a mean titer of anti-KXX antibodies or anti-monomethoxypoly(ethylene glycol) (PEG) antibodies may be performed to determine an immune reaction in the patient or subject, or to determine the efficacy of immunosuppressive therapy. In some embodiments, a mean titer of anti-KXX antibodies may be determined for a patient. In other embodiments, a mean titer of anti-monomethoxypoly(ethylene glycol) (PEG) antibodies may be determined. In some embodiments, antibody titers may be any value determined by any appropriate measurements or analyses. Antibody titers in a patient as used herein are a metric for and may indicate an immune response to a drug such as KXX. In some embodiments, an antibody titer as described herein may refer to antibodies in a patient directed against KXX or PEG. In some embodiments, an anti-KXX or anti-PEG mean antibody titer may be less than or equal to 1:7000 as a result of KXX administration. For example, an antibody titer for a patient receiving KXX therapy, either alone or in combination with MMF, may be less than or equal to about 1:100, about 1:200, about 1:300, about 1:400, about 1:500, about 1:600, about 1:700, about 1:800, about 1:900, about 1:1000, about 1:2000, about 1:3000, about 1:4000, about 1:5000, about 1:6000, about 1:7000, about 1:8000, about 1:9000, about 1:10000, or the like. In other embodiments, the antibody titers recited above may generally be found in patients who exhibit a positive response to KXX therapy (i.e., a responder). In some embodiments, a non-responder may have substantially or significantly higher antibody titers, for example, less than or equal to about 1:50000, about 1:60000, about 1:70000, about 1:80000, about 1:90000, about 1:100000, about 1:150000, about 1:200000, about 1:250000, about 1:300000, about 1:350000, about 1:400000, about 1:450000, about 1:500000, or the like. In other embodiments, such antibody titers may be determined for KXX treatment alone, or may be determined for KXX+MMF immunosuppressive therapy. Anti-KXX or anti-PEG mean antibody titers as a result of KXX and MMF immunosuppressive therapy may be beneficially maintained at or below any threshold value tolerable for the patient.

In some embodiments, anti-drug antibody titers may be reduced as a result of use of an immunosuppressive agent or therapy, such as MMF as described herein. In other embodiments, the titer of specific types of antibodies may be reduced. For example, in some embodiments, the levels or titer of any specific type of antibodies may be reduced as a result of KXX co-administered with an immunosuppressive agent or therapy, such as IgG antibodies, IgA antibodies, IgM antibodies, IgD antibodies, IgE antibodies, or combinations thereof.

In some embodiments, evaluation of antibody titers may be beneficial for patients receiving KXX, either alone, or co-administered with an immunosuppressive agent or therapy such as MMF, for the first time. In other embodiments, evaluation of antibody titers may be beneficial for patients who have developed anti-KXX antibodies, or who have been classified as non-responders to KXX treatment. Criteria for classifying a patient or subject as a responder or a non-responder are described herein elsewhere.

In some embodiments, heavier and/or younger patients may have a higher incidence of anti-KXX antibodies, or may have higher anti-KXX drug titers, than lighter and/or older patients. In other embodiments, lighter and/or older patients may have higher drug loading of KXX than heavier and/or younger patients. In some embodiments, lighter patients may have higher exposures of KXX than heavier patients. For example, in some embodiments, lighter patients may have greater than 2-fold exposure to KXX than heavier patients. In some embodiments, lower drug levels in a patient may result in anti-KXX antibodies. For example, the mean area under the curve (AUC) for lighter patients may be about 8.92 mg/L versus about 4.04 mg/L in heavier patients. In other embodiments, older patients may have higher exposures of KXX than younger patients. For example, in some embodiments, older patients may have greater than 2-fold exposure to KXX than younger patients. In some embodiments, the mean area AUC for older patients may be about 8.86 mg/L versus about 3.93 mg/L in younger patients, indicating that younger patients may have a more robust immune system. Thus, in some embodiments, additional 8-mg doses of KXX may be beneficial for younger and/or heavier patients. In other embodiments, younger and/or heavier patients may benefit from one or more loading doses of 16 mg of KXX.

In some embodiments, production of anti-drug antibodies, such as antibodies to KXX, may be reduced or eliminated by increasing the amount of KXX delivered to a patient, i.e., maintaining higher trough levels of KXX in a patient. Higher trough levels of KXX may reduce anti-drug antibody production. In some embodiments, possible dosing strategies for increasing the amount of KXX delivered to a patient, and thus producing higher trough levels in the patient, may include reducing the interval between doses of KXX, or using higher doses at the beginning of and/or during treatment. Another possible strategy for reducing anti-KXX antibodies is the use of immunomodulators, such as MMF, as described herein. Immunomodulators may reduce or eliminate an immune response to unfamiliar proteins. One of skill in the art will understand that other immunomodulators may be used with similar results, including, but not limited to, corticosteroids (i.e., prednisone), Rapimmune, myophenolate, methotrexate, or the like.

The methods disclosed herein presume that the patient is effectively receiving all of the prescribed dose. In some embodiments, depending on the age of the patient, it may be difficult to deliver a drug to a patient, for example when administering a drug to an infant and, therefore, the compliance and effectiveness of drug delivery by the patient's parent(s), guardian(s), or health care provider(s) may also be assessed.

### Definitions

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

As used in this specification and the appended claims, the singular forms “a,” “an” and “the” include plural referents unless the context clearly dictates otherwise. Thus, for example, “an active agent” refers not only to a single active agent but also to a combination of two or more different active agents, “a dosage form” refers to a combination of dosage forms as well as to a single dosage form, and the like.

Unless defined otherwise, all technical and scientific terms used herein have the meaning commonly understood by one of ordinary skill in the art to which the disclosure pertains. Specific terminology of particular importance to the description of the present disclosure is defined below.

As used herein, an “adverse event” or “AE” refers to any untoward medical occurrence associated with the use of a drug in humans, whether or not considered drug related. An AE or suspected adverse reaction is considered a “serious adverse event” or “SAE” if, in the view of either the Investigator or Sponsor, it results in any of the following outcomes: (1) Death, (2) Life-threatening: an AE is considered “life-threatening” if, in the view of either the Investigator or Sponsor, its occurrence places the subject or subject at immediate risk of death. It does not include an AE that, had it occurred in a more severe form, might have caused death; (3) Inpatient hospitalization or prolongation of existing hospitalization; (4) A persistent or significant incapacity or substantial disruption of the ability to conduct normal life functions. (5) A congenital anomaly/birth defect; (6) Important medical events that may not result in death, be life-threatening, or require hospitalization may be considered serious when, based upon appropriate medical judgment, they may jeopardize the patient or subject and may require medical or surgical intervention to prevent one of the outcomes listed in this definition. Examples of such medical events include allergic bronchospasm requiring intensive treatment in an emergency room or at home, blood dyscrasias or convulsions that do not result in inpatient hospitalization, or the development of drug dependency or drug abuse.

As used herein, “anaphylaxis” refers to a severe, acute onset allergic reaction that may occur over minutes to several hours. Anaphylaxis may involve the skin, mucosal tissue, or both, and may have one or more symptoms including, but not limited to, generalized hives, pruritus (itching), flushing, swelling of the lips, tongue, throat or uvula, shortness of breath, vomiting, lightheadedness, wheezing, hemodynamic instability, and rash or urticaria. In addition, anaphylaxis may be accompanied by at least one of the following: respiratory compromise (e.g., dyspnea, wheeze-bronchospasm, stridor, reduced peak expiratory flow, hypoxemia), and reduced blood pressure (i.e., systolic blood pressure <90 mm Hg or greater than 30% decrease from that person's baseline) or associated symptoms of end-organ failure (e.g., hypotonia [collapse], syncope, incontinence). Anaphylaxis in accordance with the disclosure is defined by the National Institute of Allergy and Infectious Disease/Food Allergy and Anaphylaxis Network (NIAID/FAAN) clinical criteria for diagnosing anaphylaxis. Anaphylaxis reactions are reported as a serious adverse event (SAE) for the present disclosure.

As used herein, “co-administration” refers to the simultaneous administration of one or more drugs with another. For example, as described herein, co-administration of KXX with MMF may refer to administration of MMF at the same time as KXX, or may refer to administration of MMF at a specific period of time before or after KXX administration. In some embodiments, MMF may be administered before KXX. In other embodiments, KXX may be administered before MMF. In other embodiments, both drugs are administered at the same time. As described herein elsewhere, co-administration may also refer to any particular time period of administration of either KXX or MMF, or both. For example, as described herein, MMF may be administered hours, days, weeks, or months before KXX treatment. In other embodiments, MMF may be administered to a patient hours, days, weeks, or months after KXX treatment. In some embodiments, co-administration may refer to any time of administration of KXX and/or MMF such that both drugs are present in the body of a patient at the same. In some embodiments, either drug may be administered before or after the other, so long as they are both present within the patient for a sufficient amount of time that the patient received the intended clinical or pharmacological benefits.

By the terms “effective amount” and “therapeutically effective amount” of an agent, compound, drug, composition or combination which is nontoxic and effective for producing some desired therapeutic effect upon administration to a subject or patient (e.g., a human subject or patient).

As used herein, a “gout flare” refers to a manifestation of physiological or biochemical symptoms of gout, which is a possible side effect or AE associated with treatment with KXX. A gout flare may produce burning itching, tingling, or stiffness in the joints, particularly the peripheral joints. An individual may also experience redness, swelling, and pain in the joints. In accordance with the disclosure, gout flares may initially increase when starting treatment with KXX. For such individuals, medications to help reduce flares may be taken regularly for the first few months after KXX is started. Prophylactic treatment for gout flares may include, but is not limited to, colchicine or non-steroidal anti-inflammatory drugs (NSAID). In some embodiments, prophylactic treatment may be given prior to an infusion of KXX, for example one week prior to treatment with KXX.

As used herein, “glucose-6-phosphate dehydrogenase (G6PD) Deficiency” or “G6PD” refers to a condition caused by an inborn error of metabolism that predisposes an individual to red blood cell breakdown. Individuals with G6PD deficiency are not included in the present study and are generally advised not to take KXX.

As used herein, “immuno-tolerance” refers to the lack of an immune response in a patient as a result of a drug treatment such as KXX. In some embodiments, establishing immune-tolerance may also refer to reducing intolerance to KXX, or to reduce or prevent loss of a response to KXX. Such loss of response may be the result of the formation of anti-drug antibodies, which may increase clearance of KXX, causing a loss of response.

As used herein, an “infusion reaction” or “IR” refers to a reaction of a patient or subject to a drug. Infusion reactions generally refer to drugs administered by intravenous (IV) infusion. most common signs and symptoms of an infusion reaction, including urticaria (skin rash), erythema (redness of the skin), dyspnea (difficulty breathing), flushing, chest discomfort, chest pain, and rash. For the present disclosure, IRs are recorded as Infusion Reaction AEs. If the IR meets the definition for “Serious” as described herein, it is also reported as an SAE. In some embodiments, an IR will be defined as any infusion-related AE or cluster of temporally-related AEs, not attributable to another cause, which occur during or within 2 hours after the infusion of pegloticase. Other AEs that occur outside of the 2-hour window following the infusion may also be categorized as an IR per the discretion of each study site. Signs and symptoms of the IR, and treatments administered, will be documented in the medical record and in the CRF. Examples of AEs not considered possible IRs include but are not limited to: laboratory abnormalities that are unlikely to have occurred during or within 2 hours following the infusion (e.g., anemia, hypercholesterolemia), gout flares, most infectious diseases, or the recurrence or worsening of a known chronic medical problem identified in the participant's medical history.

As used herein, “IR Prophylaxis” refers to a treatment regimen to prevent infusion reactions. In some embodiments, all participants will receive pre-treatment prophylaxis consisting of at least an antihistamine and corticosteroid prior to each infusion of pegloticase. In some embodiments, and in order to standardize this prophylaxis regimen, participants will take (60 mg) fexofenadine orally the night before and again on the morning of the infusion with 1000 mg/day of acetaminophen. Prior to the infusion, hydrocortisone 200 mg IV will be administered and a targeted physical exam will be performed. The name, dose, route, date, and time of administration of each prophylactic medication will be recorded in the medical record and in the CRF.

As used herein, “KXX” or “pegloticase” refers to a covalent conjugate of uricase produced by a genetically modified strain of Escherichia coli and monomethoxypoly (ethylene glycol). Although the term “uricase” or “PEGylated uricase” may be used herein to refer to a PEGylated uricase such as KRYSTEXXA®, one of skill in the art would understand that many different forms of a uricase may be known and used in accordance with the disclosure, and therefore any PEGylated uricase, such as KRYSTEXA or KXX, may be used for treatment of a patient with elevated SUA as described herein.

As used herein, the term “normal uric acid level” refers to a patient's blood plasma uric acid concentration in a range that does not cause physiological or biochemical symptoms or signs of gout. In some embodiments, a normal uric acid level may not exceed the biochemical limit of solubility. For females, a normal uric acid range may fall between about 2.4 mg/dL and about 6 mg dL, and for males, about 3.4 mg/dL to about 7 mg/dL. One of skill in the art will recognize that these values may vary slightly depending on the subject or patient, as well as on the laboratory. As used herein, the term “elevated uric acid levels” refers to refers to a patient's blood plasma or serum uric acid concentration equal to or greater than about 6 mg/dL. In some embodiments, the uric acid level in a patient may be normalized to less than about 6 mg/dL, or less than about 5 mg/dL, or less than about 2 mg/dL, following treatment with KXX, either alone or co-administered with an immunosuppressive agent or therapy. To this effect, uric acid levels can vary based on the particular testing methodology and from laboratory to laboratory.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent compound and approximately equivalent in degree. The term “pharmaceutically acceptable salts” include acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

As used herein, “prolonging” refers to extending the duration of the treatment effects of KXX therapy, either alone or co-administered with MMF. For example, as described herein, treatment of a patient with KXX co-administered with MMF, may result in a more enhanced response to the drug in the patient, resulting in a lowered SUA, when compared with treatment with KXX alone.

As used herein, “reducing” refers to a lowering or lessening, such as reducing drug intolerance to KXX in a patient. In some embodiments, co-administration of KXX and MMF results in “reducing” intolerance to KXX, indicating that the patient does not produce anti-KXX antibodies, or produces fewer anti-KXX antibodies than would be expected for a patient not receiving the same treatment. “Reducing” may also refer to a reduction in disease symptoms as a result of KXX treatment, either alone, or co-administered with MMF. “Reducing” intolerance to KXX may also be referred to herein as increasing or enhancing “immuno-tolerance.”

As used herein, “relatedness” or “causality” assessment is required for AEs (and SAEs) that occur during clinical investigations. The following terms will be used during this study:

Likely: Reasons to consider an AE likely related to treatment may include, but are not limited to the following: (1) Timing of the event relative to the administration of the investigational product. (2) Location of the AE relative to the site of investigational product administration. (3) Likelihood based on experience with similar products. (4) There is a biologically plausible explanation based on the mechanism of action or mode of delivery of the treatment. (5) The AE is repeated on subsequent treatments. (6) No other explanation is likely.

As used herein, a “severe adverse event” or “severe AE” refers to a sign, symptom, or event that causes severe discomfort to the participant and significantly affects clinical status or the ability to perform usual life activities. In some embodiments, treatment intervention may be warranted for a severe AE.

Severity in accordance with the disclosure is reported according to the following: Grade 1 (Mild)—No interference with daily activity. Grade 2 (Moderate)—Some interference with daily activity but medical intervention not required (e.g., doctor visit and/or medication); over the counter medicine permitted. Grade 3 (Severe)—Prevents daily activity and requires medical intervention (e.g., doctor visit and/or medication). Grade 4 (Potentially Life-threatening)—Emergency room visit or hospitalization.

As used herein, “subject” or “individual” or “patient” refers to any patient for whom or which therapy is desired, and generally refers to the recipient of the therapy.

As used herein, a “tolerizing dosage regimen” refers to a dosage or treatment regimen with KXX that induces immunological tolerance to the drug. A tolerizing dosage regimen prevents the loss of response to a drug in a patient by preventing the formation of anti-KXX antibodies. A tolerizing dosage regimen may also decrease the incidence of IRs associated with the drug.

The terms “treating” and “treatment” as used herein refer to reduction in severity and/or frequency of symptoms, elimination of symptoms and/or underlying cause, and improvement or remediation of damage. In certain aspects, the term “treating” and “treatment” as used herein refer to the prevention of the occurrence of symptoms. In other aspects, the term “treating” and “treatment” as used herein refer to the prevention of the underlying cause of symptoms associated with obesity, excess weight, and/or a related condition. The phrase “administering to a patient” refers to the process of introducing a composition or dosage form into the patient via an art-recognized means of introduction.

As used herein, “trough” refers to the lowest concentration of a drug in a patient before the next dose of the drug is administered. For example, trough KXX levels refer to the lowest levels of KXX in a patient before the next infusion of KXX. Trough levels may be used by clinicians or practitioners to determine the efficacy of the drug, or the response of the patient to the drug treatment. Trough levels of KXX or MMF may be determined or measured at any point during a treatment period as described herein, such as before any infusion of KXX, or before any administration of MMF.

As used herein, an “unexpected adverse event” or “unexpected AE” refers to any AE, the specificity, frequency or severity of which is not consistent with either: The known or foreseeable risk of AEs associated with the procedures involved in the research that are described in the protocol-related documents, such as the IRB-approved research protocol, any applicable investigator brochure, the current IRB-approved informed consent document, and other relevant sources of information (e.g., product labeling and package inserts); or the expected natural progression of any underlying disease or condition of the participant(s) experiencing the AE.

Unlikely: An AE with no temporal association with the study drug but rather related to other etiologies such as concomitant medications or conditions, or subject's known clinical state.

### EXAMPLES

Examples of embodiments of the present disclosure are provided in the following examples. The following examples are presented only by way of illustration and to assist one of ordinary skill in using the disclosure. The examples are not intended in any way to otherwise limit the scope of the disclosure.

### Example 1—Study Design

The disclosure describes a double-blind, placebo-controlled, multi-site study in subjects initiating pegloticase for treatment of chronic refractory gout. The purpose of the present study is to determine the efficacy and safety of using immune modulating therapy with MMF to prevent immunogenicity conferred by pegloticase.

The study will follow each participant from screening for up to six months until completion of the full study (week 24). Participants will be randomized 3:1 to either pegloticase+MMF (Peg+MMF) or to pegloticase+placebo (peg+PBO). Randomization allocation will be balanced in time and by site to achieve 24 peg+MMF and 8 peg+PBO using a double-blind design. Treatment assignment will be determined by a random number generator and stratified by site using a central randomization system to ensure the 24/8 allocation. During the first 12 weeks, participants randomized to the peg+MMF arm will receive a combination of pegloticase and MMF. Patients experience reduced immunogenicity when a loading dose of anti-proliferative agent is administered prior to a monoclonal antibody in other disease states, thus, for those randomized to the peg+MMF arm a MMF run-in will be begun at 500 mg/twice per day in the 2 weeks prior to initial pegloticase infusion. MMF (or matching PBO in the other arm) will be titrated to 1000 mg/twice per day (a standard, well-tolerated dose used in other rheumatic disease) concurrent with the first pegloticase infusion. Next, a total of up to 12 infusions of pegloticase 8 mg IV will be administered on a biweekly basis. To understand the long-term efficacy (durability) and safety of this approach, and to minimize the exposure to MMF, following the first 12 weeks of dual therapy phase, all participants will be given an additional three months of open-label pegloticase only therapy (see FIG. 2) and will be followed. Specifically, it will be assessed if the efficacy of the short course of MMF continues following discontinuation.

MMF will be purchased in bulk quantity through the UAB Investigational Drugs Pharmacy, which will also oversee the process of over-encapsulation with cellulose of the active medication and placebos, handle medication storage, distribution, and assignment of randomization sequence. The centralized management of the medication will allow maintenance of a double-blind trial. Adherence to the medication will be recorded by pill counts at the follow-up study visits and consumption of at least 80% will be required to consider the participant compliant. A non-compliant participant will continue in the study and enter analyses as mandated by statistician.

If the study drug is discontinued, unless the subject withdraws consent, the subject will be followed for the full study period and all data will be collected as scheduled. Participants that are lost to follow-up or withdraw and are not evaluable will be replaced. This may include but is not limited to the following reasons:
The subject deciding to withdraw consent for study
An intolerable adverse event (AE) as judged by study site PI and participant
The subject discontinuing acceptable birth control methods or becoming pregnant
The subject enrolling in a conflicting investigational drug trial.

### Example 2—Study Outcomes

Primary Outcomes

The primary efficacy outcome endpoint is the sustained normalization of SUA to <6 mg/dL through Week 12. This is slightly below the urate solubility threshold and this threshold has been the accepted standard for nearly all modern gout trials. Participants who achieve this endpoint will be classified as “responders.” Blood samples will be collected prior to each pegloticase infusion for measurement of SUA levels using the Beckman Coulter AU System Uric Acid procedure. Participants will be declared “non-responders” if there are two consecutive SUA measures ≥6 mg/dL within a 48-hour period. Participants who have a single SUA ≥6 mg/dL will be allowed to continue (per the prescribing information) and will be considered a responder. If the SUA goal is not maintained before or at the 12-week mark, it will be assumed that the participant has developed clinically relevant anti-pegloticase antibody and they will receive no further infusions. This assumption and subsequent discontinuation of pegloticase is consistent with the protocol of large phase III randomized controlled trials and the product label.

Safety assessments will comprise a co-primary outcome in this study and include the incidence of IRs, anaphylaxis, and the SAE/AE profile overall and potentially attributed to a combination of pegloticase with the immune modulating agent MMF. Safety assessments will include monitoring and recording of all AEs, whether drug-related or not, regular measurement of vital signs, performance of physical examinations and monitoring of hematology and blood chemistry. In the event of an AE suspected to be an IR, a blood sample will be collected at that time or the subsequent visit, centrifuged, frozen and stored for the batch evaluation of pegloticase antibodies at a future date. While we will capture gout flares as safety events only.

Secondary Outcomes

Secondary endpoints will examine anti-pegloticase Ab titers/types, different definitions of SUA level and later time points (>12 weeks) of success, and patient reported outcomes (PROs).

TABLE 1
RECIPE Study Outcomes

PRIMARY OUTCOMES
Proportion achieving and maintaining SUA <6 mg/dL
through 12 wks (responders)
Incidence and Type of Adverse Events (AEs),
Serious Adverse Events (SAEs), and
Withdrawals due to AEs, anaphylaxis,
and Infusion Reactions (IRs)
SECONDARY OUTCOMES
Anti-pegloticase antibody isotypes, specificity,
and titers
Absolute change in SUA from baseline to wk 24;
week 12 to 24
Proportion of participants with SUA <6 mg/dL
through wk 24; wk 12 to 24
PROs using PROMIS-2951,52 and Gout Impact
Score (GIS)
PROS, Patient reported outcomes;
wk, week;
SUA, serum urate;
mg/dL, milligrams/deciliter

### Example 3—Laboratory Evaluations

Hematology—Blood will be collected for measurement of hemoglobin concentration, hematocrit, erythrocyte, platelet and leukocyte counts and the differential leukocyte count at screening, and Week 24 (end of treatment) or early termination visit.

Clinical Chemistry—Blood samples will be collected prior to each pegloticase infusion for measurement of SUA levels (primary efficacy parameter) using the Beckman Coulter AU System Uric Acid procedure. In this procedure uric acid is converted to hydrogen peroxide. Hydrogen peroxide reacts with reagent to produce a chromophore which is read bichromatically at 660/800 nm. The amount of dye formed is proportional to the uric acid concentration in the sample.

Blood will be collected for measurement of transaminases (AST, ALT), alkaline phosphatase, total bilirubin, lactic dehydrogenase (LDH), creatinine, uric acid, glucose, total cholesterol, sodium, potassium, calcium, chloride, total protein, and blood urea nitrogen (BUN) at Screening and Week 24 (end of treatment) or early termination visit).

A blood sample will be obtained at Screening to evaluate Glucose-6-Phosphate Dehydrogenase (G6PD). G6PD deficiency will be an exclusion criterion.

Anti-pegloticase Antibody Assay Development—The presence or absence of antibodies will be examined in all participants at the following time point, whichever is earlier: 1) achieve non-responder status or experiencing an IR, or 2) for responders, at week 12 and week 24. Anti-pegloticase antibody titers will be determined by an enzyme-linked immunosorbent assays (ELISA) at week 12, or sooner, upon development of an immune reaction or loss of responder status, and at week 24. We will collect and store all serum samples for future batch analyses.

ELISA plates will be coated with pegloticase, recombinant uricase, or polyethylene glycol (PEG) (5 g/ml each) as the capture antigen, respectively, at 37° C. for 2 hrs. The plates will then be washed with phosphate-buffered saline-Tween-20 (PBS-T) and blocked for 60 min with PBS-T plus 3% milk (PBS-T milk). Sera will be diluted (1:30 for the anti-pegloticase or anti-uricase assay; and 1:10 for the anti-PEG assay) in PBS-T milk, will be transferred to the plates and incubated for 45 min at room temperature. The plates will then be incubated with horseradish peroxidase (HRP)-conjugated isotype-specific goat anti-human immunoglobulin-G (IgG) or goat anti-human immunoglobulin-M (IgM) (Southern Biotechnology Associates, Birmingham, Ala., USA) at 37° C. for 1 hr. Color development will be performed by using 3,3′,5,5′-tetramethylbenzidine (TMB) as the substrate. The reaction will be stopped by means of acidification and the plate will be read at 450 nm by using an Emax Precision Microplate Reader (Molecular Device, Sunnyvale, Calif., USA).

Positive Controls for the Anti-Pegloticase ELISA Assay

For the anti-pegloticase assay, a ‘positive’ ELISA response will be initially defined as an ELISA optical density A₄₅₀ greater than 3 SD above the mean for baseline pretreatment plasma samples from study subjects. Results will be further compared with those obtained from a panel of healthy control sera. Positive samples will be further validated by the specificity and sensitivity assays. Established sero-positive samples will be used as positive control for subsequent assays. The positive control for the anti-uricase antibody assay will be the monoclonal mouse anti-uricase antibody (Santa Cruz Biotechnology, Inc.). The positive control for the anti-PEG assay will be the monoclonal mouse anti-PEG antibody (Academia Sinica, Taiwan).

Determination of Anti-Pegloticase Antibody Titers and Seropositivity

For determination of Ab titers, those serum samples found to contain detectable Ab will be subjected to serial 3-fold dilutions in normal serum and analyzed in duplicate. The final titer for each sample will be defined as the highest dilution of serum that produced a mean absorbance (A₄₅₀ nm) greater than the negative cut-off value. Antibody seropositivity will be further defined as absorbance at 450 nm, >3 SD above the mean for a panel of plasma samples from naive patients.

An enzymatic/fluorescence assay will be used to quantitate pegloticase concentrations in serum. In the assay, pegloticase catalyzes the conversion of UA to allantoin, thus releasing hydrogen peroxide (H₂O₂) and carbon dioxide. In the presence of horseradish peroxidase, H₂O₂ reacts with a 1:1 stoichiometry with Amplex Ultra Red (Molecular Probes, Inc., Eugene, Oreg.) to generate the red fluorescence oxidation product, resorufin. The concentration of resorufin, which is determined by flourometry, is proportional to the amount of active pegloticase present in the serum samples. The lower limit of detection established in previous studies has been determined to be 0.6 μg/mL.

Validation of the Anti-Pegloticase ELISA Assays

Validation will include negative cut-off and cut-point factor determinations, as well as tests for intra and inter-assay precision, sensitivity, specificity and recovery, stability (bench top, freeze-thaw cycles, long-term), drug interference, prozone effect and drug competition. In previous studies the sensitivity of the anti-pegloticase Ab assay was 7.5 μg/mL for the enzyme portion and 23 ng/mL for the PEG moiety using purified rabbit anti-uricase and purified mouse anti-PEG positive controls.

Validation of anti-PEG will be carried out by determination of assay sensitivity, working range, dilutional linearity, spiking recovery, intra-assay variability, and inter-assay variability using systematic ELISA validation methods. These will be carried out with the use of selected samples diluted 1:200 or 1:20 with PBS, pH 7.2, containing 1% BSA and 0.05% Tween 20. Samples used for assay validation will be stored at −20° C. until used. Sensitivity will be determined by calculating the mean response of 10 sets of blanks and evaluating the mean plus 3 standard deviations on the standard curve. The lower limit of the working range will be defined as the sensitivity. The upper limit of the working range will be determined by the apparent value of an absorbance, which equals the mean maximum absorbance minus 3 standard deviations, as determined from the mean absorbance in 10 duplicate wells containing approximately 7.5 μg/mL of pegloticase 7.5 μg/mL for the enzyme portion and 23 ng/mL for the PEG moiety. For validation of the assay at different dilutions, at least 4 serum samples will be diluted 1:200 and 2 serum samples diluted 1:20. All serum samples will be single random samples. Dilutional linearity will be determined by evaluating each sample at its initial strength (1:200 or 1:20) and at dilutions of 1:2, 1:4, and 1:8. Spiking recovery will be determined by adding 0.0, 0.0125, 0.025, 0.050, 0.100, 0.200, and 0.400 μg/L of pegloticase to each of the diluted serum samples. Intra-assay variability will be determined by evaluating the 7 diluted serum samples 10 times within the same assay run [% CV=(standard deviations/mean)*100, where CV=coefficient of variation]. Inter-assay variability will be determined by evaluating the 7 diluted serum samples in 10 consecutive assay runs [% CV=(standard deviations/mean)*100].

Competition assays to determine the specificity of anti-pegloticase Ab and anti-uricase Ab, a variety of PEGylated proteins, including pegloticase (˜40 PEGs/protein molecule; molecular weight 545 kDa), PEG-asparaginase (˜40 PEGs/protein molecule; 340 kDa), PEG-catalase (˜40 PEGs/protein molecule; 440 kDa), PEG-chymotrypsin (˜9 PEGs/protein molecule; 70 kDa), PEG-subtilisin (˜6 PEGs/protein molecule; 57 kDa) and PEG-superoxide dismutase (˜10 PEGs/protein molecule; 82 kDa) will be used in competition assays. All of the proteins except for pegloticase will be modified with 5 kDa PEG from Sigma-Aldrich (St Louis, Mo.). Serum from anti-pegloticase sero-positive samples will be diluted 1:30 and assayed for Ab in pegloticase-coated ELISA wells in the presence or absence of 200 μg/mL of the various soluble PEGylated proteins. A mixture of lysozyme (50 μg/mL) and propylene oxide (150 μg/mL) will be used as a negative control. Also, non-PEGylated proteins will be included as the non-competitive controls. To confirm the specificity of the anti-uricase Ab assay, uricase 2 μg/mL (or 2 μg/mL lysozyme for the negative control) will be added during the serum incubation step.

### Example 4—Participant Population

We will recruit and enroll 32 adults (≥18 years of age) with SUA ≥6 mg/dL diagnosed with chronic refractory gout that have failed to normalize SUA and whose signs and symptoms are inadequately controlled with oral ULT at the maximum medically appropriate dose or the xanthine oxidase inhibitor needs to be contraindicated (FDA indication for pegloticase). Recruitment will include men and women of all races/ethnicities. Recruitment will occur at UAB and UM and approximately sixteen participants (32 overall, with approximate balance between sites) will be recruited at each site.

Inclusion Criteria

Men and women ≥18 years of age

Hyperuricemic at screening visit—SUA ≥6 mg/dL

Chronic refractory gout*, defined as subjects who failed to achieve a sustained SUA of <6 mg/dL and whose signs and symptoms are inadequately controlled with xanthine oxidase inhibitors at a medically appropriate dose or for whom these drugs are contraindicated.

2015 ACR/EULAR criteria (≥8 points)

Exclusion Criteria

Any serious acute bacterial infection (2 weeks prior to Visit 1), unless treated and complete resolved with antibiotics.

Severe chronic or recurrent bacterial infections (such as recurrent pneumonia, chronic bronchiectasis)

Current immunocompromised condition, including current or chronic treatment with immunosuppressive agents (prednisone or equivalent dose >5 mg/day)

Subjects at risk for tuberculosis (TB). Specifically, subjects with: i) current clinical, radiographic or laboratory evidence of active or latent TB; ii) a history of active TB within the last 3 years even if it was treated; iii) a history of active TB greater than 3 years ago unless there is documentation that the prior anti-TB treatment was appropriate in duration and type.

Known history of Hepatitis-B surface antigen-positive or Hepatitis B DNA positive subjects.

Known history of Hepatitis C RNA-positive subjects.

Human Immunodeficiency Virus (HIV) infection positive

G6PD deficiency (tested at Screening Visit 1)

Severe chronic renal impairment (glomerular filtration rate [GFR]<25 mL/min/1.73 m2) or currently on dialysis

Subjects having any transplant surgery requiring maintenance immunosuppressive therapy.

Non-compensated congestive heart failure, uncontrolled arrhythmia, treatment for acute coronary syndrome (myocardial infarction or unstable angina), or hospitalization for congestive heart failure within 3 months of screening or uncontrolled blood pressure (>160/100 mm Hg) at baseline (Screening Visit 1 and Week 0/Baseline visits)

Pregnant, planning to become pregnant, breast-feeding, or not on an effective form of birth control

Prior treatment with pegloticase, another recombinant uricase, or concomitant therapy with a polyethylene glycol (PEG)-conjugated drug

Known allergy to pegylated products or history of anaphylactic reaction to a recombinant protein or porcine product

MMF treatment is contraindicated or considered inappropriate.

Recipient of an investigational drug within 4 weeks prior to study drug administration or plans to take an investigational agent during the study, or any exclusionary drugs.

Current liver disease as determined by alanine transaminase ALT or aspartate transaminase (AST) levels >3 times upper limit of normal

Currently receiving treatment for ongoing cancer, excluding non-melanoma skin cancer

History of malignancy within 5 years other than skin cancer or in situ carcinoma of cervix

Uncontrolled hyperglycemia with a plasma glucose value >240 mg/dL at screening

Diagnosed osteomyelitis

Individuals with hypoxanthine-guanine phosphoribosyl-transferase (HGPRT) deficiency such as Lesch-Nyhan and Kelley-Seegmiller syndrome

Not good candidate for the study based on opinion of the Investigator (e.g., cognitive impairment) that might create undue risk to the participant or interfere with the participant's ability to comply with the protocol requirements, or to complete the study.

### Example 5—Study Procedures and Assessments

This study will be coordinated by the University of Alabama at Birmingham (UAB). All study visits and procedures will be performed at the United States, at UAB, the University of Michigan, and up to 6 additional to be named clinical study sites. Approximately n=8 participants per site will be enrolled at the UAB and UM study sites, and approximately n=4 participants will be recruited at each to be identified study site. Enrollment will be competitive between sites.

Frequency of study visits and clinical evaluations can be found in Table 2. Following the first 12-week dual therapy phase, participants will be given an additional three months of open-label pegloticase only therapy (see FIG. 2) and will be followed to better understand the long-term efficacy (durability) of this approach, which is essential for future studies. Specifically, we will assess the durability of the short course of MMF, continues over the next 12 weeks without using MMF.

Participants will be seen at screening, baseline, and every two weeks thereafter for pegloticase infusion and evaluation. Blood samples prior to each pegloticase dose will allow measurement of SUA, comprehensive metabolic panel, and complete blood count (CBC) will allow appropriate measurement for the co-primary safety and secondary outcomes with respect to MMF (Table 1).

Screening Visit

The screening visit will take approximately 1 hour to complete. Potential participants will be screened to determine if they satisfy all inclusion and exclusion criteria. Men and women 18 years of age or older will be invited to proceed with informed consent (IC) and enroll in the study. At the screening visit, the study objectives will be explained to potential participants. After all questions raised by a potential participant are answered, and before any protocol-specified screening procedures are initiated, they will be offered the IC for the screening evaluation. A copy of the signed and dated IC form must be provided to the participant.

After IC is obtained, a 6-digit participant number will be assigned. The first 3 digits of each participant number will represent the site and the last 3 will be unique for each participant at each site. All screening procedures must be completed and eligibility criteria met prior to start of immunemodulating therapy and pegloticase infusions. Basic demographic information and reason(s) for exclusion must be completed on the specified case report form (CRF) pages for all participants who signed an ICF, but never received pegloticase. During the screening visit, the following procedures will be performed, and information will be obtained to determine eligibility to continue in this research study:

Review inclusion/exclusion criteria

ICF

Date of birth

Self-reported race/ethnicity

Medical history that might preclude study participation

Gout history and symptom severity

Medication review
Medication history (including use of over the counter medications [eg. aspirin], use of other prescription medications including gout medications) Dietary supplement/vitamin use
Vital signs

Physical exam includes, but is not limited to: Eye, Head, Ears, Nose, and Throat Exam (HENT), and Neck; Cardiovascular; Dermatological; Respiratory; Gastrointestinal; Musculoskeletal; Neurologic; Integumentary; VS/Measurements

Gout Flare/assessment

PROs (eg. PROMIS-29 & GIS instrument)

Blood draw

Screening Visit Laboratory

CBC with diff

### HIV1 and 2 Antibody Screen

### IgG

### SUA

Pregnancy test for premenopausal women

### Comprehensive Metabolic Panel (CMP)

### G6PD

Blood sample for serum banking

Vital signs will consist of heart rate, respiratory rate, blood pressure (noting the position in which it was obtained), and body temperature (taken either orally or aurally). All measurements of pulse rate and blood pressure should be made after approximately 5 minutes of rest. Focused history and physical examination: Information collected will include date of birth, self-reported race/ethnicity (defined as in previous studies investigating its role in rheumatic diseases, gout history, medication history (including use of aspirin, gout medications), weight, and height.

Assessments for presence of tophi will be conducted as well as gout history and symptom severity.
Document the number of gout flares in the last 6 months and 12 months and the most recent occurrence. Patients with gout flares can enter the study if the flare treatment is discontinued 1 week prior to the first dose of pegloticase.
Document the presence and/or history of gout-related kidney disease.

Physical examinations will be performed by body system at Screening and Week 24 (end of treatment) or early termination visit in the pegloticase dosing phase. Significant findings prior to the administration of pegloticase must be recorded in the patient's medical record and included on the Medical History in the CRFs. Significant findings that occur after administration of pegloticase which meet the definition of an AE must be recorded in the medical record and on the Adverse Events CRF page.

All women of childbearing potential must use an effective form of birth control during this study and for 30 days after completion of the study. Acceptable methods of birth control include hormonal control methods, inter-uterine device, a double-barrier method (diaphragm with spermicide, condom with spermicide) or abstinence. All male participants will be cautioned to use proper birth control methods with their partners during the course of the study in which MMF is received.

Laboratory: SUA, CMP, CBC with diff, and pregnancy test for premenopausal women. Additionally, a sample will be collected at screening, Visit 1, Visit 7, and Visit 13 for evaluation of anti-pegloticase Ab. Serum samples will be collected and prepared (centrifuged and frozen) for transport to the clinical research lab in the Division of Clinical Immunology and Rheumatology at the University of Alabama at Birmingham. All lab samples will be discarded if the participant is deemed not eligible for the study.

Run-in Visit

Obtain and record vital signs, including pulse rate, sitting blood pressure, and body temperature

Review/update concomitant medications

Gout Flare/tophus assessment

Blood draw

Laboratory
CBC with diff
SUA
Pregnancy test for premenopausal women
CMP

Dispense 2 week course of MMF (500 mg/twice per day) or placebo, with dosing instructions

Gout Flare Prophylaxis

Subjects will be placed on a prophylactic regimen of colchicine or NSAID to prevent gout flares, unless medically contraindicated or not tolerated, and will receive this prophylaxis for at least two weeks prior to the first administration of pegloticase. Gout flare prophylaxis will continue for the duration of the study unless medically contraindicated or not tolerated.

Visit 1 (Baseline-0 Weeks)

Visit 1 will occur within 2 weeks of the run-in visit.

The markers for the study primary and secondary outcomes will be collected at each visit. Other data will be collected as needed for safety monitoring.

Obtain and record vital signs, including pulse rate, sitting blood pressure, and body temperature

Review/update concomitant medications

Assess any AEs, and record

Gout Flare assessment

Assess compliance (via pill count) and dispense course of MMF (1000 mg/twice per day) or PBO, with dosing instructions

PROs (PROMIS-29 & GIS instrument)

Targeted Physical Exam and joint assessment

Laboratory
CBC with diff
IgG
SUA
Pregnancy test for premenopausal women
CMP
Serum collection

Gout Flare Prophylaxis

IR Prophylaxis/Assess for IRs

Administer pegloticase infusion, per site guidelines

In addition to gout flare prophylaxis all participants will receive infusion prophylaxis throughout the study (e.g., fexofenadine (60 mg PO) the night before and fexofenadine (60 mg PO) and acetaminophen (1000 mg) the morning of the infusion; and hydrocortisone (200 mg IV) immediately prior to the infusion).

Visit 2 (2 Weeks)

Obtain and record vital signs, including pulse rate, sitting blood pressure, and body temperature

Targeted Physical Exam

Review/update medical history concomitant medications

Assess any AEs, and record

Gout Flare assessment

Assess compliance (via pill count)

Laboratory
CBC with diff
CMP
SUA
Pregnancy test for premenopausal women

Gout Flare Prophylaxis

Administer pegloticase infusion, per site guidelines

IR Prophylaxis/Assess for IRs

Visit 3 (4 Weeks)

Obtain and record vital signs, including pulse rate, blood pressure, and body Temperature

Targeted Physical Exam and joint assessment

Review/update concomitant medications

Assess any AEs, and record

Gout Flare assessment

PROs (PROMIS-29 & GIS instrument)

Assess compliance (via pill count) and dispense course of MMF (1000 mg/twice per day) or PBO, with dosing instructions

Laboratory Assessments
CBC with diff
CMP
SUA
Pregnancy test for premenopausal women

Gout Flare Prophylaxis

IR Prophylaxis/Assess for IRs

Administer pegloticase infusion, per site guidelines

Visit 4 (6 Weeks)

Obtain and record vital signs, including pulse rate, blood pressure, and body temperature

Targeted Physical Exam and joint assessment

Review/update concomitant medications

Assess any AEs, and record

Gout Flare assessment

Assess compliance (via pill count)

Laboratory Assessments
CBC with diff
IgG
SUA
Pregnancy test for premenopausal women
CMP

Gout Flare Prophylaxis

IR Prophylaxis/Assess for IRs

Administer pegloticase infusion, per site guidelines

Visit 5 (8 Weeks)

Obtain and record vital signs, including pulse rate, blood pressure, and body temperature

Targeted Physical Exam and joint assessment

Review/update concomitant medications

Assess any AEs, and record

Gout Flare assessment

PROs (PROMIS-29 & GIS instrument)

Assess compliance (via pill count) and dispense course of MMF (1000 mg/twice per day) or PBO, with dosing instructions

Laboratory Assessments
CBC with diff
CMP
SUA
Pregnancy test for premenopausal women

Gout Flare Prophylaxis

IR Prophylaxis/Assess for IRs

Administer pegloticase infusion, per site guidelines

TABLE 2
Schedule of Planned RECIPE Visits and Evaluations

| | | | | | | | | | | | | | | Month 6 | |
|---|---|---|---|---|---|---|---|---|---|---|---|---|---|---|---|
| | | | | | | | | | | | | | | | V13 |
| | Screening/Run-in | | | | | | | | | | | | | | ((Early |
| | Screen | Run-in | Month 1 | | | Month 2 | | Month 3 | | Month 4 | | Month 5 | | | Termination/ |
| Shady | S1 | S2 | V1 | V2 | V3 | V4 | V5 | V8 | V7 | V8 | V9 | V10 | V11 | V12 | Closeout- |
| procedures | (−4 wks) | (−2 wks) | (0 wks) | (2 wks) | (4 wks) | (6 wks) | (8 wks) | (10 wks) | (12 wks) | (14 wks) | (16 wks) | (18 wks) | (20 wks) | (0 wks) | 24 wks) |
| ICF | X | | | | | | | | | | | | | | |
| Medical history | X | | | | | | | | | | | | | | |
| Physical exam | X | | | | | | | | X | | | | | | |
| Vital signs* | X | X | X | X | X | X | X | X | X | X | X | X | X | X | X |
| PROS | X | | X | | X | | X | | X | | X | | X | | X |
| Update current medical | | X | X | X | X | X | X | X | X | X | X | X | X | X | X |
| conditions and concomitant | | | | | | | | | | | | | | | |
| medications | | | | | | | | | | | | | | | |
| Assess AEs | | | X | X | X | X | X | X | X | X | X | X | X | X | X |
| Dispense | | X | X* | | X | | X | | | | | | | | |
| MMF/PBO | | (500 mg/ | (1 g/ | | (1 g/ | | (1 g/ | | | | | | | | |
| | | 2x per day) | 2x per day) | | 2x per day) | | 2x per day) | | | | | | | | |
| Drug accountability | | X | X | X | X | X | X | X | X | X | X | X | X | X | |
| Targeted PE | | | X | X | X | X | X | X | | X | X | X | X | X | X |
| CBC with diff | X | X | X | X | X | X | X | X | X | X | X | X | X | X | X |
| HIV1 and 2 | X | | | | | | | | | | | | | | |
| Antibody Screen | | | | | | | | | | | | | | | |
| IgG | X | | X | | | X | | | X | | | | | | |
| SUA (Pre Infusion | X | X | X | X | X | X | X | X | X | X | X | X | X | X | X |
| Weeks 0-24⁺⁺) | | | | | | | | | | | | | | | |
| Pregnancy | X | X | X | X | X | X | X | X | X | X | X | X | X | X | X |
| testing | | | | | | | | | | | | | | | |
| CMP | X | X | X | X | X | X | X | X | X | X | X | X | X | X | X |
| G6PD | X | | | | | | | | | | | | | | |
| Biospecimen storage£ | X | | X | | | | | | X | | | | | | X |
| Adminster gout flare | | X | X | X | X | X | X | X | X | X | X | X | X | X | |
| prophylaxis^{±} | | | | | | | | | | | | | | | |
| Infusion reaction | | | X | X | X | X | X | X | X | X | X | X | X | X | |
| prophylaxis ≠ | | | | | | | | | | | | | | | |
| Adminster pegloticase | | | X | X | X | X | X | X | X | X | X | X | X | X | |

G6PD = glucose-6-phosphate dehydrogenase (All participants will be tested for G6PD); CMP = Comprehensive metabolic profile. hematology will include hemoglobin concentration and hematocrit; erythrocyte, platelet, and leukocyte counts; differential leukocyte count, serum chemistry will include transaminases, alkaline phosphatase, total bilirubin, lactic dehydrogenase (LDH), uric acid, glucose, total cholesterol, sodium, potassium, calcium, chloride, total protein, and blood urea nitrogen (BUN); CBC with diff = Complete Blood Count with Differentiation; IR = infusion reaction, MMF = mycophenolate mofetil, PROs = patient reported outcomes, SUA = serum uric acid. For the pegloticase dosing phase, Dose 1 will be scheduled once it has been confirmed that the participant has been on gout flare prophylaxis for at least a week and is able to take the prophylaxis IR drugs prior to the first visit. Doses 2-6 will be scheduled within 14 ± 3-days post prior dose; *Includes sitting blood pressure, heart rate, respiratory rate, and body temperature. Vital signs should be collected before study, drug infusion or pre-medications, and every 30 mins during the infusion of study drug. ≠ IR prophylaxis should be self-administered the night before ((60 mg PO) fexofenadine) and the morning of the day of pegloticase dosing ((60 mg PO) fexofenadine plus (1000 mg PO) acetaminophen); and hydrocortisone (200 mg IV) immediately prior to the infusion); ⁺⁺The sUA results from UAB and UM will be used in determining if participant receives pegloticase infusion; ‡The participant should begin a regime of colchicine or NSAID gout flare prophylaxis at least 1 week before the first dose of pegloticase and it should continue for the duration of pegloticase therapy. ¥500 mg/2x per day will be dispensed at run-in. Depending on tolerability will be increased to 1 gm/.2x per day 1. £Serum samples will be collected for analysis of AB and in the event of IR.

Visit 6 (10 Weeks)

Obtain and record vital signs, including pulse rate, blood pressure, and body temperature

Targeted physical exam

Review/update concomitant medications

Assess any AEs, and record

Gout Flare Assessment

Assess drug compliance (via pill count)

Laboratory Assessments
CBC with diff
CMP
SUA
Pregnancy test for premenopausal women

Gout Flare Prophylaxis

IR Prophylaxis/Assess for IRs

Administer pegloticase infusion, per site guidelines

Visit 7 (12 Weeks)

Obtain and record vital signs, including pulse rate, blood pressure, and body temperature

Review concomitant medications, and record

Assess any AEs, and record

Gout Flare Assessment

PROs (PROMIS-29 & GIS instrument)

Assess drug compliance (via pill count)

Physical Exam and detailed joint assessment
Eye, HENT, and Neck
Cardiovascular
Respiratory
Gastrointestinal
Musculoskeletal
Neurologic
Integumentary
VS/Measurements

Laboratory Assessments
CBC with diff
IgG
SUA
Pregnancy test for premenopausal women
CMP
Serum collection

Gout Flare Prophylaxis

IR Prophylaxis/Assess for IRs

Administer pegloticase infusion, per site guidelines

Following completion of Visit 7 participants will continue on pegloticase for an additional twelve weeks without immune modulating therapy to evaluate the longer term benefits of this approach. In this phase participants will continue with pegloticase infusions every 2 weeks.

Visit 8 (14 Weeks)

Obtain and record vital signs, including pulse rate, blood pressure, and body temperature

Targeted physical exam

Review/update concomitant medications

Assess any AEs, and record

Gout Flare Assessment

Laboratory Assessments
CBC with diff
SUA
Pregnancy test for premenopausal women
CMP

Gout Flare Prophylaxis

IR Prophylaxis/Assess for IRs

Administer pegloticase infusion, per site guidelines

Visit 9 (16 Weeks)

Obtain and record vital signs, including pulse rate, blood pressure, and body temperature

Targeted Physical Exam and joint assessment

Review/update concomitant medications

Assess any AEs, and record

Gout Flare Assessment

PROs (PROMIS-29 & GIS instrument)

Laboratory Assessments
CBC with diff
SUA
Pregnancy test for premenopausal women
CMP

Gout Flare Prophylaxis

IR Prophylaxis/Assess for IRs

Administer pegloticase infusion, per site guidelines

Visit 10 (18 Weeks)

Obtain and record vital signs, including pulse rate, blood pressure, and body temperature

Targeted Physical Exam and joint assessment

Review concomitant medications, and record

Assess any AEs, and record

Gout Flare Assessment

Laboratory Assessments
CBC with diff
SUA
Pregnancy test for premenopausal women
CMP

Gout Flare Prophylaxis

IR Prophylaxis/Assess for IRs

Administer pegloticase infusion, per site guidelines

Visit 11 (20 Weeks)

Obtain and record vital signs, including pulse rate, blood pressure, and body temperature

Targeted Physical Exam and joint assessment

Review concomitant medications, and record

Assess any AEs, and record

Gout Flare assessment

PROs (PROMIS-29 & GIS instrument)

Laboratory Assessments
CBC with diff
SUA
Pregnancy test for premenopausal women
CMP

Gout Flare Prophylaxis

IR Prophylaxis/Assess for IRs

Administer pegloticase infusion, per site guidelines

Visit 12 (22 Weeks)

Obtain and record vital signs, including pulse rate, blood pressure, and body temperature

Targeted Physical Exam and joint assessment

Review concomitant medications, and record

Assess any AEs, and record

Gout Flare assessment

Laboratory Assessments
CBC with diff
SUA
Pregnancy test for premenopausal women
CMP

Gout Flare Prophylaxis

IR Prophylaxis/Assess for IRs

Administer pegloticase infusion, per site guidelines.

Visit 13 (Discontinuation/Closeout Visit 24 Weeks)

Visit 13 will serve as the study closeout or discontinuation visit. Participants will complete a final physical exam, blood draw, and update their medical history since enrollment.

Obtain and record vital signs, including pulse rate, blood pressure, and body temperature

Review concomitant medications, and record

Assess any AEs, and record

PROs (PROMIS-29 & GIS instrument)

Physical exam
Eye, HENT, and Neck
Cardiovascular
Respiratory
Gastrointestinal
Musculoskeletal
Neurologic
Integumentary
VS/Measurements

Laboratory Assessments
CBC with diff
SUA
Pregnancy test for premenopausal women
CMP
Serum collection

Unscheduled Visit—

An unscheduled Visit will be conducted in the event of a suspected AE thought to be severe.

Obtain and record vital signs, including pulse rate, blood pressure, and body temperature

Review concomitant medications, and record

Assess any AEs, and record

Physical exam includes, but is not limited to
Eye, HENT, and Neck
Cardiovascular
Respiratory
Gastrointestinal
Musculoskeletal
Neurologic
Integumentary
VS/Measurements

Laboratory Assessments
CBC with diff
IgG
SUA
CMP

### Example 6—Informed Consent (IC)

Informed Consent Procedures—The process of IC will be carried out by one of the study physicians in conjunction with the study coordinator/research assistant involved in the screening visit after the participant appears to meet the pre-screening criteria. During the screening visit, the ICF will be read by the study participant and then each section will be explained by the research study coordinator obtaining consent. During this process, individuals will be informed of all aspects of the study so that they can make an informed decision. Participants will then confirm their willingness to participate in the research study by signing the informed ICF. The participant will be given as much time as they need to read and ask questions about the consent form. The individual or participant's legally authorized representative will be informed that he/she is not obligated to participate in the study and that it is strictly voluntary and that he/she may withdraw from the study at any time and that withdrawal of consent will not affect his/her subsequent medical treatment or relationship with the treating physician.

The IC process will ensure that there is no penalty for not participating in a clinical trial and that treatment will not be compromised if individuals do not participate or if they cease participation at any time. By signing the consent form, the participant authorizes the use of their personal health information, indicates that they understand the study and its benefits and risks, and agrees to all other aspects of the study outlined in the form.

After the participant has signed the ICF, the Principal Investigator (PI), and the study coordinator conducting the visit must each sign and date the ICF. A signed version of the ICF will be kept by the study staff in the study binder and an additional copy of the consent form will also be given to the participant to keep.

The IC document contains the following:

Disclosure of relevant information to prospective participants about the research

The participant's comprehension of the information

The participant's voluntary agreement to participate in a research study without coercion or undue influence

Complete disclosure of any appropriate alternative procedures and their risks and benefits

Disclosure of the extent of confidentiality that will be maintained

Statement of compensation and/or medical treatment available if injury occurs

Name, address, and telephone number of the participants

Informed Consent Changes—If there is a change in any of the study procedures that may affect the participant, the ICF will be revised and approved by the necessary Institutional Review Boards (IRBs). Any participants enrolled in the study prior to a change in procedures will sign the amended consent form at the next physical encounter with the recruitment site. Per National Institute of Health (NIH) policy, the signed consent forms will be scanned into the electronic database and kept as part of the study record for at least 7 years after completion of the study and stored electronically in servers housed within the UAB School of Medicine. Participants can withdraw their consent and revoke their data authorization at any time by informing the local study coordinator or investigator. For participants that withdraw their consent, no further data will be collected via survey or annual assessment and the recruitment site will be notified. Per FDA guidance, their existing study data will be maintained to ensure scientific validity of the study.

### Example 7—Medications and Dosing

Pharmacy—The UAB Investigational Drug Service (IDS) and the University of Michigan Clinical Research Pharmacy are responsible for the storage and preparation of both pegloticase. The UAB IDS will be responsible for the storage and over encapsulation of mycophenolate mofetil, and placebo pills for this study. The labeled study drugs will be picked up by a research assistant to dispense to study participants. A drug log will be used to track the study drug from pharmacy to study participant. MMF will be purchased in bulk quantity through the UAB Investigational Drugs Pharmacy, which will also oversee the process of over-encapsulation with cellulose of the active medication and placebos, handle medication storage, distribution, and assignment of randomization sequence. The centralized management of the medication will allow maintenance of a double-blind trial. Adherence to the medication will be recorded by pill counts at the follow-up study visits and consumption of at least 80% will be required to consider the participant compliant. A non-compliant participant will continue in the study and enter analyses as mandated by statistician. Further questions about pharmacy activities can be directed to: Rebecca Quinn, PharmD. IDS Pharmacy, University of Alabama at Birmingham Hospital, 205-934-7191 and Helen Tamer, PhD University of Michigan Clinical Research Pharmacy, 734-936-8210.

Pegloticase—Pegloticase, a clear, colorless, sterile solution in phosphate-buffered saline intended for IV infusion after dilution, will be supplied by Horizon Pharma, PLC. Pegloticase is commercially available in the US in a single-use, 2 mL glass vial with a Teflon coated (latex-free) rubber injection stopper. Each mL of pegloticase contains 8 mg of uricase protein conjugated to 24 mg of 10 kDa monomethoxypoly(ethylene glycol). Excipients include disodium hydrogen phosphate dihydrate, sodium chloride, sodium dihydrogen phosphate dehydrate, and water for injection.

All participants in the study will receive pegloticase at the same dose of 8 mg administered IV every 2 weeks for a total of 6 infusions over a 12-week treatment period, and over an additional 12-week pegloticase opt-in follow-up period (per standard of care).

Packaging and Clinical Supplies—Study drug pegloticase will be provided by Horizon Pharma, PLC.

Storage and Return—Before preparation for use, pegloticase will be stored in the carton, maintained under refrigeration between 2° C. and 8° C. (36° F. and 46° F.), protected from light, and will not be shaken or frozen. Investigational clinical supplies will be received by a designated person at the study site, handled and stored safely and properly, and kept in a secured location to which only the Investigator and designated assistants have access. Clinical supplies will be dispensed only in accordance with the protocol. We will keep accurate records of the clinical supplies received and, the amount dispensed for each participant, and the amount remaining at the conclusion of the study. We will mark the label of any vials that are not to be used with a large “X,” and document the reason for rejecting them on the drug accountability log. In accordance with good pharmacy practice, gloves will be worn during preparation of the dose.

The study sites will maintain an inventory of drug supplies received and dispensed. UAB will provide forms to document all inventory transactions. Upon completion or termination of the study, all clinical pegloticase supplies (used and unused), will be destroyed with written certification confirming destruction within sixty (60) days of study completion or expiration of the study drug.

Preparation—Vials will be visually inspected for particulate matter and discoloration before administration, whenever solution and container permit. Vials will not be used if either is present. Using appropriate aseptic technique, 1 mL of pegloticase will be withdrawn from the vial into a sterile syringe. Any unused portion of product remaining in the vial will be discarded. Syringe contents will be injected into a single 250 mL bag of 0.45% or 0.9% Sodium Chloride Injection, United States Pharmacopeia (USP) for IV infusion and will not be mixed or diluted with other drugs. The infusion bag containing the dilute pegloticase solution will be inverted a number of times to ensure thorough mixing, but will not be shaken.

Pegloticase-diluted in infusion bags is stable for 4 hours at 2° C. to 8° C. (36° F. to 46° F.) and at room temperature (20° C. to 25° C., 68° F. to 77° F.); however, the diluted solution will be stored under refrigeration, not frozen, protected from light, and used within 4 hours of dilution. Before administration, the diluted solution of pegloticase will be allowed to reach room temperature. Pegloticase in a vial or IV infusion fluid will never be subjected to artificial heating.

Administration—Pegloticase will be administered as an admixture of 8 mg in 250 mL of 0.45% or 0.9% Sodium Chloride Injection, USP for IV infusion over a target infusion time of 120 minutes by gravity feed or infusion pump. Pegloticase will not be administered as an IV push or bolus. Standardized IR prophylaxis consisting of pre-treatment with antihistamines and corticosteroids will accompany each infusion. The drug name, dose, and timing of these prophylactic medications will be recorded.

Participants will not be fasting on the day of infusion; they will be encouraged to have a snack or normal meal 1 hour before, or immediately after, the infusion. Prior to pegloticase infusion participants will receive infusion prophylaxis (e.g., oral fexofenadine (60 mg) the night before and fexofenadine (60 mg/PO) and acetaminophen (1000 mg/PO) the morning of the infusion; and hydrocortisone IV (200 mg) immediately prior to the infusion).

In a patent IV site, using tubing with no in-line filter, infuse the drug preparation over approximately 120 minutes (within ±15 minutes) while the participant is under close observation for any signs of distress. Administration of drug will be immediately discontinued if respiratory distress, agitation, chest or back pain, urticaria, or another clinically significant event occurs during infusion. If the AE meets the definition of an SAE, the infusion may not be restarted under any circumstances. A SAE will be reported within 24 hours or sooner to the Data Safety Monitoring Board (DSMB). If the AE does not meet the definition of an SAE, the site PI may make the decision to re-start the infusion depending upon the nature and severity of the AE.

Infusions subsequent to an infusion-related reaction in an individual participant may be given in a larger volume of diluent, not to exceed 500 mL. In such a case, the infusion duration will also be extended to a minimum of 3 hours. The total volume and duration of infusion will be captured in the medical record and CRF.

As a precaution, emergency equipment will be readily available to treat a possible hypersensitivity reaction, and will include drugs that would be used to treat an anaphylactic reaction. Personnel fully trained in advanced cardiopulmonary resuscitation and in the use of the emergency equipment will be readily available during, and for 1 hour after, the infusion. At the end of the infusion, the IV line will be flushed with 10 mL of normal saline to assure the full dose is administered. As IRs can occur after completion observation of participants for approximately an hour post-infusion will be performed.

Mycophenolate Mofetil (MMF)—

MMF (brand name CellCept®), the immune modulator for the study, is the 2-morpholinoethyl ester of moiety mycophenolic acid (MPA), and is an inosine monophosphate dehydrogenase (IMPDH) inhibitor. The chemical name for MMF is 2-morpholinoethyl (E)-6-(1,3-dihydro4-hydroxy-6-methoxy-7-methyl-3-oxo-5-isobenzofuranyl)-4-methyl-4-hexenoate. It has an empirical formula of C23H31NO7.

MMF will be purchased by UAB from Besse Medical (West Chester Township, Ohio).

Packaging and Clinical Supplies:

Labeling—Blinded study drug labeling will be annotated with the protocol number by the research pharmacist at the University of Alabama at Birmingham.

Storage and return of MMF—The Coordinating Center pharmacy at the University of Alabama at Birmingham will maintain an inventory of drug supplies received and dispensed.

Handling and Disposal—MMF has demonstrated teratogenic effects in rats and rabbits. MMF tablets should not be opened or crushed. Avoid inhalation or direct contact with skin or mucous membranes of the powder contained in MMF tablet/capsules.

Preparation—MMF capsules include croscarmellose sodium, magnesium stearate, povidone (K-90) and pregelatinized starch. The MMF capsule shells contain black iron oxide, FD&C blue #2, gelatin, red iron oxide, silicon dioxide, sodium lauryl sulfate, titanium dioxide, and yellow iron oxide. All MMF capsules will be over-encapsulated with cellulose to match compounded placebo. Favorable pharmacokinetic properties of the encapsulation process have been consistently noted with other substances.

Administration—Patients will be instructed that oral dosage tablets (over encapsulated MMF and PBO) will be administered on an empty stomach (1 hour before or 2 hours after meals) to avoid variability in MPA absorption. If a dose is missed, it will be administered as soon as it is remembered. If it is close to the next scheduled dose, the participant will be instructed to skip the missed dose and resume at the next regularly scheduled time; thus, participants will be instructed not to double a dose to make up for a missed dose.

Concomitant Medications—Concomitant medications are defined as drug or biological products other than the study drug(s) taken by a participant during the clinical trial. This includes other prescription medications (including preventive vaccines), over-the-counter medications, herbal medications, vitamins, and food supplements.

A comprehensive list of participant's concomitant medications will be collected at baseline and at each visit. This will include the name of the drug/vitamin/supplement, dose, route of administration, start and stop dates, and the reason for which the medication was taken. All medications will be listed by participant using the generic name(s) of the drug/vitamin/supplement.

Severe/Serious adverse events related to the use of a concomitant drug/vitamin/supplement will be documented on the appropriate AE CRF.

Gout Flare Prophylaxis—All participants will receive prophylactic treatment to reduce the risk of acute gout flares, unless medically contraindicated or not tolerated as noted in the FDA-approved pegloticase full prescribing information. The participant will begin a regime of colchicine (0.6 mg/day) or NSAID prophylaxis at least 1 week before the first dose of pegloticase and it will continue for the duration of pegloticase therapy. Colchicine prophylaxis will not be interrupted during the course of the clinical trial unless medically contraindicated or if the participant becomes intolerant of colchicine, regardless of whether a gout flare occurs.

Gout Flare Treatment—An increase in gout flares is frequently observed upon initiation of anti-hyperuricemic therapy, including treatment with pegloticase. Participants will be instructed to contact the site within 12 hours of the onset of symptoms. Gout flares will be confirmed through questioning or direct observation. All participants who experience a gout flare during the study will be prescribed anti-inflammatory treatment (e.g., corticosteroids, NSAIDs, colchicine) as deemed clinically indicated by the study physician.

Colchicine will be prescribed in a medically appropriate dose range of 0.6 to 1.8 mg/day, usually dosed as 0.6 mg PO/three times per day unless reduced dosing is necessitated by renal insufficiency or gastrointestinal intolerance. The precise dose and regimen of colchicine will be individualized for each participant by the investigators, such as in the case of renal insufficiency where colchicine is appropriately started at 0.6 mg/day and increased to tid as tolerated.

### Example 8—Statistical Methods

The co-primary aims of our initial double-blind, placebo controlled proof-of-concept study are to 1) assess the feasibility of a short course of immune modulating therapy with daily mycophenolate mofetil (MMF). We will start to test the hypothesis that MMF for 12 weeks will safely attenuate immunogenicity conferred by pegloticase as determined by the proportion of participants achieving and maintaining an SUA <6 mg/dL through 12 weeks, compared to concurrent controls, and assessing the incidence and types of adverse events/infusion reactions potentially associated with a dual therapy regimen of pegloticase and MMF. After 12 weeks of co-administration, all participants will continue on pegloticase for an additional 12 weeks without combination MMF therapy to evaluate the longer term benefits and safety of this approach; and 2) Assess the incidence and types of adverse events/infusion reactions.

Data Management—The DCC has extensive experience in data management in over approximately 15 active national/international collaborative studies. The data management systems and approaches employed for RECIPE will be based on existing, highly successful platforms. The MITS Suite (a distributed data management system) consists of the MITS Studio (electronic Data Entry Management System [eDEMS] Authoring Tool) and the Testing Framework. The clinical centers are responsible for the entry and management of data from their own center, providing cost-effective (as it removes practically all of the query process) higher quality data.

Validation of data—Validation of data will be done using the MITS Suite, which contains mechanisms to ensure accuracy, reliability, and the ability to discern invalid or altered records.

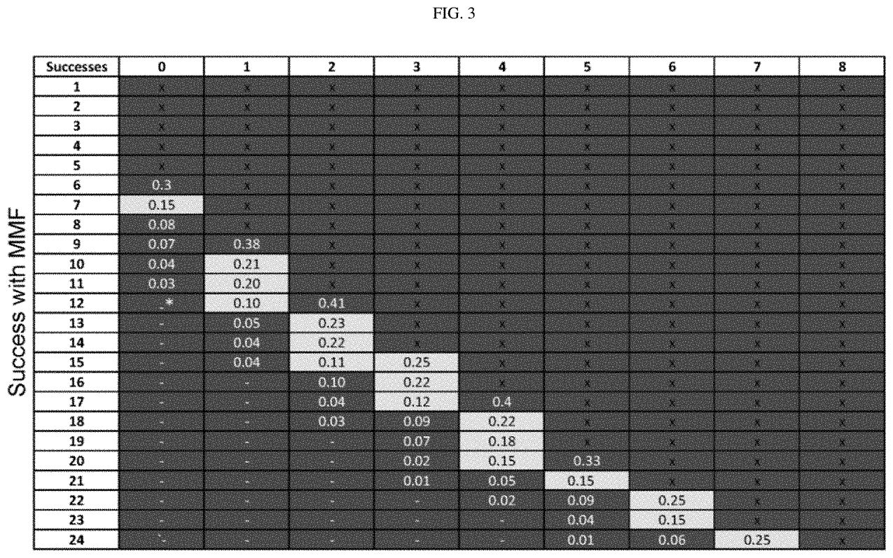

Data Analysis—Primary Aims—One of the primary objectives of this feasibility pilot study is to improve the efficacy and safety of pegloticase, a PEGylated recombinant uricase, used in the treatment of refractory gout, and to determine if there is an overall reduction in immunogenicity leading to increased responders to pegloticase when MMF is co-administered in adults with chronic refractory gout. A decision table was prepared indicating when sufficient evidence is present to move forward to a full scale clinical trial (FIG. 3). Comparison may be made comparing the success rates in the peg+MMF arm (N=24) versus peg+PBO (N=8). The area in green is the area that we will lead us to recommend continuing to test the treatment approach in a full-scale study assuming safety. This green area represents the area that achieves a significant (2 tailed p<0.10) Fisher's exact test that peg+MMF is better than pegloticase alone. They yellow area represents an achievement of a 2-tailed p<0.25. If the results end up in the yellow or red areas, we will examine the Ab assays to determine if there is a clear significant difference in immune response. Based on these sample sizes, the mean difference in Ab titer level needs to be at least 1 standard deviation unit apart to achieve 73% Power.

Nevertheless, this would be a large difference for a clinical outcome variable, but for a marker such as Ab titers this is not unreasonable. Should the titer distributions not be consistent with normality assumptions, nonparametric analyses using a Wilcoxon test will be used, since the titer levels are already measured on a log scale precluding a simple transformation to achieve normality. Participants before the 12 weeks primary endpoint that do not tolerate MMF, or are lost to follow-up, withdraw, or are otherwise not evaluable will be counted as failures but not non-responders in sensitivity analyses.

In the event of missing antibody data, imputations will be considered to assure data completeness for our analyses using PROC MI and PROC MIANALYZE procedures with 5 replicates per value. All analyses will be conducted using SAS (V9.4, Cary, N.C.).

Data Analysis—Secondary Aims and Safety Aims.

The secondary and safety aims are to: 1) Determine the 6 month durability of immune modulation after discontinuation of the short course of MMF by: a) assessing the absolute change in SUA from baseline to Week 24, and Week 12 to Week 24, and b) determining the proportion of participants with SUA <6 mg/dL through 24 weeks, and Week 12 to Week 24; 2) Identify and characterize the pegloticase immune response by immunoglobulin isotypes (IgG and IgM), specificities, and antibody titer, and 3) Examine patient reported outcomes (PROs) using the NIH supported Patient Reported Outcomes Measurement Information System (PROMIS) and Gout Impact Scale (GIS) instruments.

### Example 9—Adverse Events (AEs)

This is a Phase II, double-blind, placebo controlled examining two medications (pegloticase and MMF) that are already FDA approved and have been in clinical use for over 5 years, but are not commonly co-administered. An AE is defined as any untoward event whether or not considered related to the use of pegloticase or MMF. Any worsening (i.e. any clinically significant adverse change in frequency or intensity) of a preexisting condition which is temporally associated with the use of pegloticase or MMF is also considered an AE. Abnormal laboratory values or test results constitute AEs only if they induce clinical signs or symptoms or require therapy, and are recorded on the AE CRF under the signs, symptoms or are associated with diagnoses associated with them. Screening conditions will not be considered AE; however, worsening of a preexisting condition may be considered an AE. We will start collecting AEs at our baseline, Visit 1.

The safety events of interest in assessing study risks and benefits are IRs and a co-primary study outcome. Participants will be followed for the occurrence of IR and secondary outcomes of interest events at each study visit. Supplementing the data collected during these visits will be information collected regarding participant reported health-related quality of life (QOL), and for improved, near real-time assessment of outcome events. Non-severe/serious events that are expected according to previous experience with the study drugs (pegloticase, MMF) as described in the protocol, consent materials, or any approved product labelling will also be collected. We will report all severe/serious AEs according to appropriate authority (e.g., FDA, IRB) in compliance with guidelines and regulations.

Expected AEs Associated with KXX

An AE that is not an unexpected AE is an infusion reaction and rarely anaphylaxis. Safety assessments in this study include an evaluation of the frequency and severity of IRs and anaphylaxis. During pre-marketing controlled clinical trials, infusion reactions were reported in 26% of patients treated with pegloticase 8 mg every 2 weeks. During pre-marketing controlled clinical trials, anaphylaxis was reported with a frequency of 6.5% of patients treated with pegloticase. For the purposes of this study, these events shall be defined as follows:

IR not attributable to another cause that occurs during or within 2 hours after the infusion of pegloticase will be defined as an AE. Other cases that occur outside of the 2-hour window may also be categorized as an IR as per site PI discretion.

Anaphylaxis will be defined using the National Institute of Allergy and Infectious Diseases (NIAID)/Food Allergy and Anaphylaxis Network (FAAN) criteria: acute onset of an illness (minutes to several hours) with involvement of the skin, mucosal tissue, or both (e.g., generalized hives; pruritus or flushing; urticarial, and angioedema (of lips, tongue, or uvula) and at least one of the following: Hypotension (i.e., systolic blood pressure <90 mm Hg or >30% decrease from that person's screening) or associated symptoms of end-organ failure (e.g., hypotonia [collapse], syncope, incontinence; and respiratory compromise (e.g., dyspnea, bronchospasm, stridor, reduced peak expiratory flow, hypoxemia).

Pegloticase has not been formally studied in patients with congestive heart failure, but some patients in clinical trials have experienced exacerbation. Patients who have diagnosed congestive heart failure will not be enrolled in the study. It is common for potent urate lowering therapies to lead to acute attacks of gout. Other uncommon symptoms reported in at least 5% patients treated with pegloticase that may occur during the study period are ecchymoses, sore throat, constipation, chest pain, and vomiting.

Expected AEs Associated with MMF

Gastrointestinal: Nausea and vomiting (RA: 12%), diarrhea;

Hematologic & oncologic: Leukopenia (renal transplant: >50%; RA: 28%), neoplasia (renal transplant 3% (other than lymphoma), 0.5% (lymphoma)), thrombocytopenia;

Hepatic: Hepatotoxicity, increased serum alkaline phosphatase, increased serum bilirubin, increased serum transaminases;

Infection: Increased susceptibility to infection (renal transplant 20%; RA <1%; includes bacterial, fungal, protozoal, viral, opportunistic, and reactivation of latent infections)

Expected AEs Associated with Colchicine

Participants taking colchicine for gout flare prophylaxis may experience gastrointestinal intolerance which may lead to nausea, persistent diarrhea, and/or gastrointestinal bleeding. The most commonly reported side effects for the prophylaxis of gout was diarrhea (23%) and pharyngolaryngeal pain (3%).

Other AEs associated with colchicine include: Neutropenia, leading to an increased risk of infection; Anemia; Myalgia or myositis; Alopecia; Pruritus; Neuropathy; Oligospermia.

While taking colchicine participants should avoid eating grapefruit and Seville oranges or drinking grapefruit juice or Seville orange juice. These can increase their chances of getting serious side effects.

Serious Adverse Event (SAE) Criteria

A SAE is any AE occurring at any dose that results in any of the following outcomes: Death; Is life-threatening (places the participant, in the view of the site PI, at immediate risk of death from the AE as it occurred); Inpatient hospitalization or prolongation of existing hospitalization (hospitalization is defined as an inpatient admission, regardless of length of stay, even if hospitalized as a precautionary measure for continued observation); A permanent, persistent, or significant disability (substantial disruption of the ability to conduct normal life functions). A medically significant AE that may jeopardize the participant and may require medical or surgical intervention to prevent one of the outcomes listed in this definition

Events NOT considered to be Severe or Serious AEs are: Hospitalization for treatment, which was elective or pre-planned, for a pre-existing condition that did not worsen; Treatment on an emergency, outpatient basis for an event NOT fulfilling any of the definitions of serious given above and NOT resulting in hospital admission; An event that, had it occurred in a more severe form, might have caused death; A sign, symptom, or event that is noticeable but easily tolerated; An event does not significantly influence performance or prevent the participant from carrying on with usual life activities.

Infusion Reaction Prophylaxis

All participants will receive pre-treatment prophylaxis consisting of at least an antihistamine and corticosteroid prior to each infusion of pegloticase (see Table 3). In order to standardize this regimen, participants will take (60 mg) fexofenadine orally the night before and again on the morning of the infusion with 1000 mg/PO of acetaminophen. Prior to the infusion, hydrocortisone 200 mg IV will be administered and a targeted physical exam will be performed. The name, dose, route, date, and time of administration of each prophylactic medication will be recorded in the medical record and in the CRF.

TABLE 3
Infusion Reaction Prophylaxis

| | | Following arrival at |
|---|---|---|
| Night Before Infusion | Morning of Infusion | infusion clinic |
| Participant takes: | Participant takes: | Abbreviated physical |
| Fexofenadine vv | Fexofenadine v | examination to include: |
| (60 mg) PO | (60 mg) PO | Dermatological - |
| | Acetaminophen | noting any rashes |
| | (1000 mg) PO | Chest - noting breath |
| | | sounds |
| | | Vital signs |
| | | Start IV and administer |
| | | hydrocortisone (200 mg) |
| | | Initiate drug infusion |

### Example 10—Discontinuation and Participant Withdrawal

Due to the risk of anaphylaxis and IRs being higher in patients who have lost therapeutic response, participants with two consecutive SUA levels equal to or above 6 mg/dL shall be classified as a non-responder and discontinued from the study. Investigators will obtain a pre-dose SUA sample for all patients and review results to verify the SUA level is <6 mg/dL prior to infusion.

Participants can withdraw their consent and discontinue the study at any time by informing the study coordinator. Following OHRP guidance and recommendations on this topic (hhs.gov/ohrp/policy/subjectwithdrawal.html#), if a subject decides to withdraw, we will inquire as to whether the subject wishes to withdraw from all components of the trial or only from the primary intervention. If the latter, we will encourage continuation on follow-up data collection activities for which the subject previously gave consent. These data may prove very useful Moreover, we will document each case of withdrawal, whether it was based on the decision of the participant or investigator (e.g., non-compliance), and whether the withdrawal was from all study components or only the primary intervention.

If the study drug is discontinued, unless the subject withdraws consent, the subject will be followed for the full study period and all data will be collected as scheduled. Participants that are lost to follow-up or withdraw and do not provide data informative to the primary study and will be replaced. This may include but is not limited to the following reasons:
The subject deciding to withdraw consent for study
An intolerable AE as judged by study site PI and participant
The subject discontinuing acceptable birth control methods or becoming pregnant
The subject enrolling in a conflicting investigational drug trial

In the event a participant is lost to follow-up, withdraws consent and discontinues the study before the end of week 12 and is therefore unable to contribute to the primary outcome, the enrolling site will be afforded the opportunity to enroll a replacement participant. Attempts will be made to schedule an early end of study assessment in the case of study drug discontinuation.

As stated above, premenopausal women will have a pregnancy test before the study starts and again throughout the study. If participants suspect that they may have become pregnant during the study, the study coordinator will contact the study PI immediately and the PI or Study Coordinator will instruct the participant to stop taking all study medication. If it is confirmed that the participant is pregnant, they will be withdrawn from the study. The study PI will schedule a follow-up visit and may choose to follow the outcome of the pregnancy. If it is discovered that participants are breastfeeding, they are not eligible to participate in the study and their participation will be discontinued immediately.

Based on the known safety profiles of pegloticase and MMF and the procedures we have in place, we believe it is very unlikely, but possible that we could witness one severe AE related to infection. We will institute a stopping rule for safety re-evaluation that would occur if we register more than one of such severe adverse reaction (eg. infection that leads to hospitalization). We would then stop the study to comprehensively review safety and our study protocols in conjunction with the DSMB. Other severe adverse reaction or deaths may or may not be related to the study and stopping or discontinuation of the study will be considered on an individual basis.

### Example 11—Results

A total of 42 patients were screened, of which 35 were enrolled in the study, and the remaining 7 patients were excluded for inclusion/exclusion criteria or declined to participate. Of the 35 patients enrolled, 3 withdrew after randomization but before the first pegloticase infusion. The remaining 32 participants were included in analyses. Patients were randomized into either the pegloticase+MMF group (n=22) or the pegloticase+placebo (PBO) group (n=10). Patient demographics are provided in Table 4.

TABLE 4
Demographics of Patient Population

| | |
|---|---|
| North | North |

| | | | | | | |
|---|---|---|---|---|---|---|
| | UAB | Michigan | Altoona | Mississippi | Alabama | Total |
| | (N = 17) | (N = 7 ) | (N = 7) | (N = 2) | (N = 2) | (N = 35) |
| Demographics | N (Col. %) | N (Col. %) | N (Col. %) | N (Col. %) | N (Col. %) | N (Col. %) |

Gender

| | | | | | | | | | | | | |
|---|---|---|---|---|---|---|---|---|---|---|---|---|
| Female | 0 | (0%) | 3 | (42.9%) | 0 | (0%) | 0 | (0%) | 1 | (50%) | 4 | (11.4%) |
| Male | 17 | (100%) | 4 | (57.1%) | 7 | (100%) | 2 | (100%) | 1 | (50%) | 31 | (88.6%) |

Race

| | | | | | | | | | | | | |
|---|---|---|---|---|---|---|---|---|---|---|---|---|
| American Indian or | 0 | (0%) | 0 | (0%) | 1 | (14.3%) | 0 | (0%) | 0 | (0%) | 1 | (2.9%) |
| Alaska Native | | | | | | | | | | | | |
| Asian | 0 | (0%) | 0 | (0%) | 0 | (0%) | 0 | (0%) | 0 | (0%) | 0 | (0%) |
| Black or African- | 3 | (17.6%) | 2 | (28.6%) | 0 | (0%) | 0 | (0%) | 0 | (0%) | 5 | (14.3%) |
| American | | | | | | | | | | | | |
| Native Hawaiian or | 1 | (5.9%) | 0 | (0%) | 0 | (0%) | 0 | (0%) | 0 | (0%) | 1 | (2.9%) |
| other Pacific | | | | | | | | | | | | |
| Islander | | | | | | | | | | | | |
| White | 13 | (76.5%) | 5 | (71.4%) | 6 | (85.7%) | 2 | (100%) | 2 | (100%) | 28 | (80%) |

Of the pegloticase+MMF group, 19 patients completed the 12-week assessment, and 3 patients were discontinued from the study for treatment failure (SUA >6). Of the 19 patients in the pegloticase+MMF group who completed the 12-week assessment, 14 patients went on to complete the 24-week assessment, and 5 patients were discontinued from the study for treatment failure (n=3), adverse events (n=1), or lack of participation (n=1). Three additional patients were discontinued during the follow-up period, leaving 11 patients in the group who completed the study.

Of the pegloticase+PBO group, 4 patients completed the 12-week assessment, and 6 were discontinued from the study either for infusion reactions (n=3) or treatment failure (n=3). Of the 4 patients in the pegloticase+PBO group who completed the 12-week assessment, 2 patients went on to complete the 24-week assessment, and 2 patients were discontinued from the study for treatment failure (n=1), or concerns from an ongoing COVID-19 pandemic (n=1). One additional patient was discontinued during the follow-up period due to COVID-19 concerns, leaving 1 patient in the group who completed the study.

A total of 12 patients completed the full study protocol (36 weeks), and 23 patients experienced treatment failure or were discontinued prior to completing full protocol.

Patient baseline characteristics are provided in Table 5.

TABLE 5
Baseline Characteristics

| | Participants | | Pegloticase + | | Pegloticase + | | |
|---|---|---|---|---|---|---|---|
| | (n = 32) | | MMF (n = 22) | | PBO (n = 10) | | P-value |
| Gender (%) | | | | | | | |
| Men | 28 | (88) | 19 | (86) | 9 | (90) | 0.99 |
| Age in years, mean (SD) | 55.1 | (9.6) | 55.0 | (9.3) | 55.4 | (10.6) | 0.91 |
| 2015 ACR/EULAR criteria | 32 | (100) | 22 | (100) | 10 | (100) | N/A |
| points ≥8 (%) | | | | | | | |
| 2015 ACR/EULAR criteria | 13.6 | (2.7) | 13.5 | (2.8) | 13.8 | (2.7) | 0.88 |
| points, mean (SD) | | | | | | | |
| Gout flare history | | | | | | | |
| Flare within last year (%) | 20 | (63) | 15 | (68) | 5 | (50) | 0.44 |
| Number of flares last | 1 | (0, 2) | 1 | (0, 2) | 1 | (0, 1) | 0.28 |
| year, median (Q1, Q3) | | | | | | | |
| Age at diagnosis, years, | 41.3 | (13.8) | 40.9 | (14.7) | 42.1 | (12.6) | 0.99 |
| mean (SD) | | | | | | | |
| Duration of Gout, years, | 13.4 | (9.0) | 13.3 | (9.8) | 13.4 | (7.4) | 0.82 |
| mean (SD) | | | | | | | |
| Gout impact score at | 45.4 | (8.0) | 45.6 | (8.1) | 44.8 | (8.1) | 0.79 |
| screening (range: 0-96) | | | | | | | |
| Mean (SD) | | | | | | | |
| PROMIS Scores at screening | | | | | | | |
| Pain intensity T-Score, | 53.0 | (9.5) | 54.1 | (9.3) | 50.6 | (10.1) | 0.35 |
| mean (SD) | | | | | | | |
| Physical function T-Score, | 36.0 | (7.3) | 36.1 | (7.8) | 35.7 | (6.5) | 0.88 |
| mean (SD) | | | | | | | |
| Urate lowering agents | | | | | | | |
| Allopurinol | 19 | (59) | 13 | (59) | 6 | (60) | 0.99 |
| Febuxostat | 5 | (16) | 4 | (18) | 1 | (10) | 0.99 |
| Acute gout therapy | | | | | | | |
| Colchicine | 14 | (44) | 9 | (41) | 5 | (50) | 0.71 |
| NSAID | 21 | (66) | 16 | (73) | 5 | (50) | 0.44 |
| Corticosteroids | 6 | (19) | 4 | (18) | 2 | (20) | 0.99 |
| Alcohol Consumption | | | | | | | |
| (# of drinks) | | | | | | | |
| 0 | 14 | (44) | 11 | (50) | 3 | (30) | 0.62 |
| 1-2 | 11 | (34) | 7 | (32) | 4 | (40) | |
| >2 | 7 | (22) | 4 | (18) | 3 | (30) | |
| Pain score History | 4.0 | (3.8) | 4.5 | (4.0) | 2.8 | (3.3) | 0.36 |
| (0 to 10, 10 being the | | | | | | | |
| worst), mean (SD) | | | | | | | |
| Serum urate, mg/dL mean (SD) | 7.6 | (2.2) | 7.6 | (2.2) | 7.6 | (2.3) | 0.97 |
| Serum urate | | | | | | | |
| ≤6 mg/dL | 9 | (28) | 7 | (32) | 2 | (20) | 0.68 |
| >6 mg/dL | 23 | (72) | 15 | (68) | 8 | (80) | |
| eGFR, mean (SD) | 81.3 | (24.1) | 82.7 | (26.8) | 78.0 | (17.3) | |
| <90 (mL/min/1,73 m²) | 24 | (75) | 15 | (68) | 9 | (90) | 0.48 |
| ≥90 (mL/min/1,73 m²) | 8 | (25) | 7 | (32) | 1 | (10) | 0.38 |
| Presence of Tophi | 28 | (88) | 19 | (86) | 9 | (90) | 0.99 |
| BMI (%) | | | | | | | |
| 25 to <30 | 4 | (13) | 3 | (14) | 1 | (10) | 0.6316 |
| 30 to <45 | 14 | (44) | 8 | (36) | 6 | (60) | |
| 35 to <40 | 8 | (25) | 7 | (32) | 1 | (10) | |
| 40 to <45 | 4 | (13) | 3 | (14) | 1 | (10) | |
| ≥45 | 2 | (6) | 1 | (4) | 1 | (10) | |
| Comorbidities, Yes | | | | | | | |
| Pneumonia | 6 | (19) | 4 | (18) | 2 | (20) | 0.9999 |
| Diabetes | 4 | (13) | 3 | (14) | 1 | (10) | 0.9999 |
| Cerebral Vascular Accident | 2 | (6) | 2 | (9) | 0 | (0) | 0.9999 |
| Peripheral vascular disease | 1 | (3) | 1 | (5) | 0 | (0) | 0.9999 |
| High blood pressure | 25 | (78) | 18 | (82) | 7 | (70) | 0.6479 |
| Coronary artery disease | 3 | (9) | 1 | (5) | 2 | (20) | 0.2238 |
| Other heart disease | 10 | (31) | 5 | (23) | 5 | (50) | 0.2168 |
| Cancer | 2 | (6) | 2 | (9) | 0 | (0) | 0.9999 |
| Metabolic syndrome | 1 | (3) | 0 | (0) | 1 | (10) | 0.3125 |
| Dyslipidemia | 12 | (38) | 8 | (36) | 4 | (40) | 0.9900 |
| Kidney stones | 9 | (28) | 4 | (18) | 5 | (50) | 0.0960 |
| Chronic kidney disease | 3 | (9) | 1 | (5) | 2 | (20) | 0.2239 |
| Autoimmune Disease | 3 | (9) | 3 | (14) | 0 | (0) | 0.5343 |
| Allergy to any other medication | 8 | (25) | 5 | (23) | 3 | (30) | 0.6808 |

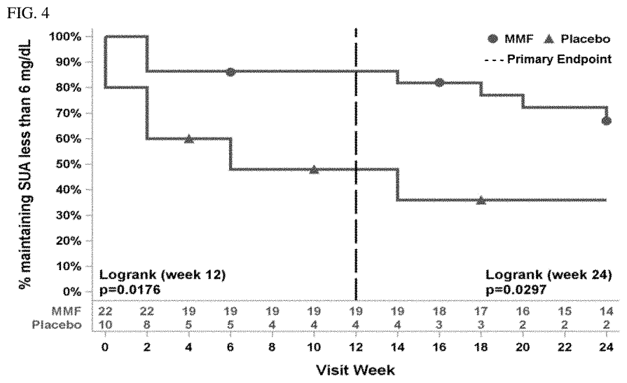

For this study, 32 subjects received at least one dose of pegloticase and were included in modified intention to treat analyses. 19 of 22 subjects (86% response rate) in the pegloticase+MMF arm achieved the primary outcome at 12 wks vs. 4 of 10 subjects (40% response rate) in the pegloticase+placebo arm (p-value 0.01). At week 24, a 68% response rate was seen for the pegloticase+MMF group vs a 30% response rate seem for the pegloticase+placebo group. The p-values for these response rates were significant, particularly noting that the pegloticase+MMF group only got MMF for the run-in period and the first 12 weeks with pegloticase. Primary and secondary efficacy outcomes are provided in FIG. 4 and Tables 6-7 below.

TABLE 6
Primary Efficacy Outcome

| | Pegloticase + | Pegloticase + | Difference | |
|---|---|---|---|---|
| | MMF | PBO | between | |
| | % [95% CI] | % [95% CI] | two groups | P- |
| Primary Outcome | (N) | (N) | % [95% CI] | value |
| Serum uric | 86% [65, 97] | 40% [12, 74] | 46% [13, 80] | 0.01 |
| acid <6 mg/dL | (19/22) | (4/10) | | |
| at week 12 | | | | |

TABLE 7
Secondary Efficacy Outcomes

| | Pegloticase + | Pegloticase + | Difference | |
|---|---|---|---|---|
| | MMF % | PBO % | between two | |
| | [95% CI] | [95% CI] | groups % | P- |
| Secondary Outcome | (N) | (N) | [95% CI] | value |
| Serum uric acid < 6 mg/dL at | 86% [65, 97] | 40% [12, 74] | 46% [13, 80] | 0.01 |
| week 12 | (19/22) | (4/10) | | |
| Serum urate < 6 mg/dL | 15/22 (68) | 3/10 (30) | (38) [4 to 73] | 0.0623 |
| (Baseline to week 24) | [49 to 88] | [2 to 58] | | |
| Serum urate < 6 mg/dL | 15/19 (79) | 3/4 (75) | (4) [−42 to 50] | 0.9999 |
| (week 12 to week 24) | [54 to 94] | [19 to 99] | | |
| | Median | Median | | |
| | (Q1, Q3) | (Q1, Q3) | Mean | |
| | mean | mean | Difference | P |
| | (SD), (N) | (SD) (N) | [95% CI] | Value |
| Absolute sUA change up to | 7.5 (1.8, 8.9) | 3.1 (1.4, 5.7) | 1.5 [−1.8, 4.7] | 0.4083 |
| week 24 | 5.7 (4.0) (22) | 4.2 (4.1) (9) | | |
| Absolute sUA change from | 0.1 (0, 5.2) | 0.05 (0, 0.2) | 1.8 [−1.3, 5.0] | 0.4767 |
| week 12 to week 24 | 1.9 (3.0) (19) | 0.1 (0.1) (4) | | |
| *Proportion w/ serum uric | 15/22 (68) | 3/10 (30) | (38) | 0.0623 |
| acid < 6 mg/dL up to week 24 | [49 to 88] | [2 to 58] | [4 to 73] | |
| Proportion w/ Serum uric | 15/19 (79) | 3/4 (75) | (4) | 0.9999 |
| acid < 6 mg/dL from week 12 | [54 to 94] | [19 to 99] | [−42 to 50] | |
| to week 24 | | | | |
| *PROMIS Scores at 12 | | | | 0.8851 |
| weeks (the higher score | | | | 0.8851 |
| the more severity). | | | | |
| Pain intensity T-Score, | N = 19, 48.8 | N = 3, 47.2 | 1.5 | |
| mean (SD) | (9.2) | (6.2) | [−10.1, 13.1] | |
| Median (Q1, Q3) | 49.4 | 49.4 | | |
| | (43.5, 57.5) | (20.2, 52.1) | | |
| Physical function T- | N = 19, 37.2 | N = 3, 34.3 | 2.8 | |
| Score, | (11.0) | (6.6) | [−11.0, 16.7] | |
| mean (SD) | | 32.1 | | |
| | | (29.1, 1.8) | | |
| Median (Q1, Q3) | 34.4 | | | |
| | (29.1, 45.3) | | | |
| Pain score History (0 to 10, | 5.4 (3.0) | 5.5 (3.1) | −0.1 | 0.9999 |
| the higher had worst | 5.5 (3.0, 8.0) | 4.5 (3.5, 7.5) | [−4.0, 3.8] | |
| imaginable pain) | (10) | (4) | | |
| mean (SD) | | | | |
| Median (Q1, Q3) | | | | |
| Revised gout impact score at | 43.7 (6.9) | 40.7 (5.5) | 3.0 | 0.41 |
| 12 weeks (The higher score | 44.0 | 38.0 | [−5.8, 11.8] | |
| the more severity, range: 0-96) | (39.0, 49.0) (19) | (37.0, 47.0)(3) | | |

Adverse Events (AEs)

The estimated rates of AEs per month were similar between the pegloticase+MMF (0.3) and the pegloticase placebo (0.4) groups. The placebo group had a greater percentage of infusion reactions (30% vs. 0%). Tables 8-10 provide the AEs observed in this study. Eleven (11) reported infusion reaction symptoms were associated with three participants in the placebo (PBO) group.

TABLE 8
Adverse Events

| | |
|---|---|
| Pegloticase + MMF | Pegloticase + PBO |
| (Freq. of Events = 44) | (Freq. of Events = 10) |

Adverse Event

| | | | | |
|---|---|---|---|---|
| Mild N (%) | 21 | (48%) | 5 | (50%) |
| Moderate N (%) | 20 | (45%) | 4 | (40%) |
| Severe N (%) | 0 | (0%) | 0 | (0%) |
| Serious Adverse | 3 | (7%) | 1 | (10%) |
| Event N (%) | | | | |
| Life Threatening | 0 | (0%) | 0 | (0%) |
| N (%) | | | | |
| Death N (%) | 0 | (0%) | 0 | (0%) |

TABLE 9
Most Commonly Reported Adverse Events

| | Pegloticase + | | Pegloticase + | |
|---|---|---|---|---|
| | MMF | | PBO | |
| | (Freq. of | | (Freq. of | |
| | Events = 44) | | Events = 10) | |
| Any Adverse Event (AE) | 41 | (93%) | 9 | (90%) |

Most commonly reported

| | | | | |
|---|---|---|---|---|
| Gout Flares | 8 | (13%) | 2 | (3%) |
| Cardiac Disorders (e.g., | 2 | (3%) | 1 | (2%) |
| hypertension, chest pain) | | | | |
| Gastrointestinal Disorders | 4 | (9%) | 1 | (2%) |
| (e.g., (diarrhea, upset | | | | |
| stomach, nausea, dehydration) | | | | |
| Infections (e.g., UTI, | 2 | (3%) | 0 | (0%) |
| paronychia) | | | | |
| Musculoskeletal and Connective | 11 | (18%) | 0 | (0%) |
| Tissue Disorders | | | | |
| (other muscle issues) | | | | |
| Respiratory (allergies; | 4 | (6%) | 0 | (0%) |
| congestion; upper resp. tract | | | | |
| infection; asthma exacerbation) | | | | |
| Skin Disorders (poison ivy; rash; | 2 | (3%) | 1 | (2%) |
| tongue irritation) | | | | |
| Other (lab abnormality, anxiety, | 5 | (8%) | 0 | (0%) |
| neurological, oral pain, vascular, | | | | |
| injury, elevated ALT/AST, fatigue, | | | | |
| eye blurriness, dizziness, abnormal | | | | |
| lab value AST, and | | | | |
| decrease in hemoglobin/hematocrit) | | | | |

Table 10 shows data relating to Serious Adverse events (SAEs).

TABLE 10
Serious Adverse Events (SAEs)

| SAE | Pegloticase + MMF | | Pegloticase + PBO | |
|---|---|---|---|---|
| Mild N (%) | 0 | (0%) | 0 | (0%) |
| Moderate N (%) | 0 | (0%) | 0 | (0%) |
| Severe N (%) | 0 | (0%) | 0 | (0%) |
| Serious Adverse | 3 | (7%) | 1 | (25%) |
| Event N (%) | | | | |
| Life Threatening N (%) | 0 | (0%) | 0 | (0%) |
| Death N (%) | 0 | (0%) | 0 | (0%) |

As a result of this study, it was determined that use of MMF was associated with a statistically significant and clinically meaningful impact on the proportion of subjects achieving and maintaining a sUA <6 mg/dL. Short-term concomitant use of MMF with pegloticase was generally well tolerated. In addition, the estimated rates of AE's were generally similar between the study groups.

It can be concluded that co-administration of an immunomodulatory agent could mitigate this loss of efficacy and concern for drug-related toxicity. The study described herein suggests an innovative approach to customize therapy in refractory patients and to ameliorate infusion reactions.

Throughout the study, certain amendments were implemented. Protocol amendments included a restriction of body weight, a reduction of visits during long-term follow-up, removal of targeted physicals during long-term follow-up.

What is claimed is:

1. A method of treating gout in a patient having a serum uric acid level of ≥6 mg/dL comprising:
administering mycophenolate mofetil (MMF) to said patient at a dose of 500 mg twice per day orally for a period of 2 weeks prior to the first administration of a PEGylated uricase;
co-administering a PEGylated uricase and MMF to said patient using a dosage regimen comprising a dose of 8 mg of the PEGylated uricase intravenously every 2 weeks for a total of 12 doses; and a dose of 1000 mg MMF twice per day orally, wherein the co-administered MMF is administered concurrently with each administration of the PEGylated uricase;
administering 8 mg of the PEGylated uricase at a dosage of 8 mg intravenously every 2 weeks for a total of 12 doses.

2. A method of reducing or preventing loss of response to a PEGylated uricase and prolonging the urate lowering effect comprising co-administration of the PEGylated uricase at a dosage of 8 mg intravenously every 2 weeks and mycophenolate mofetil (MMF) at a dosage of 1000 mg twice per day orally to a patient having a serum uric acid level of ≥6 mg/dL prior to PEGylated uricase treatment initiation;
wherein the co-administration of the PEGylated uricase and MMF result in the serum uric acid level being normalized relative to a patient not receiving co-administration of the PEGylated uricase and MMF immunosuppressive therapy.

3. The method of claims 1 or 2, further comprising a prophylactic regimen of colchicine for a period of at least 2 weeks prior to the first administration of the PEGylated uricase.

4. The method of any of claims 1 to 3, wherein the SUA levels of the patient are determined prior to each dose of the PEGylated uricase.

5. The method of any of claims 1 to 3, further comprising measuring one or more of trough PEGylated uricase levels, anti-PEGylated uricase antibody levels, and anti-monomethoxypoly(ethylene glycol) (PEG) antibody levels, prior to each dose of the PEGylated uricase after the first dose.

6. The method of claim 2, further comprising measuring hematology and liver function on a weekly basis or every 2 weeks during treatment.

7. The method of any of claims 2 to 6, wherein said co-administration of the PEGylated uricase and MMF results in normalization of the serum uric acid level in the patient relative to a patient not receiving co-administration of the PEGylated uricase and MMF.

8. The method of any of claims 1 to 7, wherein the serum uric acid level is reduced to less than 6 mg/dL as a result of co-administration of the PEGylated uricase and MMF.

9. The method of any of claims 1 to 8, wherein the serum uric acid level is reduced to less than 5 mg/dL as a result of co-administration of the PEGylated uricase and MMF.

10. The method of any of claims 1 to 9, wherein the serum uric acid level is reduced to less than 2 mg/dL as a result of co-administration of the PEGylated uricase and MMF.

11. The method of any of claims 1 to 10, wherein the incidence of infusion reaction, gout flare, or anaphylaxis is reduced as a result of co-administration of the PEGylated uricase and MMF.

12. The method of any of claims 1 to 11, wherein the level of MMF metabolite is increased relative to a patient not receiving co-administration of the PEGylated uricase and MMF.

13. The method of claims 1 or 2, further comprising measuring one or more of peripheral joint urate deposition volume and inflammatory volume.

14. The method of claim 13, wherein peripheral joint urate deposition volume is reduced in the patient relative to a patient not receiving co-administration of the PEGylated uricase and MMF treatment.

15. The method of claim 14, wherein peripheral joint urate deposition volume is determined by dual-energy computed tomography (DECT) scanning.

16. The method of claim 13, wherein inflammatory volume is reduced in the patient relative to a patient not receiving co-administration of the PEGylated uricase and MMF treatment.

17. The method of claim 16, wherein inflammatory volume is determined by Dynamic Contrast Enhanced-Magnetic Resonance Imaging (DCE-MRI) or MRI without contrast, or both.

18. The method of any of claims 1 to 17, wherein:
the mean titer of anti-PEGylated uricase antibodies is less than or equal to 1:7000 as a result of the PEGylated uricase and MMF treatment.

19. The method of any of claims 1 to 18, wherein the serum uric acid level is normalized by week 12 after the PEGylated uricase and MMF treatment begins.
